(19) United States
(12) Patent Application Publication
(10) Pub. No.: US 2014/0050697 A1
(43) Pub. Date: Feb. 20, 2014

(54) ANALYSIS OF HCV GENOTYPES

(71) Applicant: Vertex Pharmaceuticals Incorporated, Cambridge, MA (US)

(72) Inventors: Douglas John Bartels, North Liberty, IA (US); Ann Dak-Yee Kwong, Cambridge, MA (US); Tara Lynn Kieffer, Brookline, MA (US)

(73) Assignee: Vertex Pharmaceuticals Incorporated, Cambridge, MA (US)

(21) Appl. No.: 13/911,410

(22) Filed: Jun. 6, 2013

Related U.S. Application Data
(63) Continuation of application No. 13/036,262, filed on Feb. 28, 2011, now abandoned.
Application No. 13/036,262 is a continuation of application No. PCT/US2009/055385, filed on Aug. 28, 2009.
(60) Provisional application No. 61/092,503, filed on Aug. 28, 2008.

(51) Int. Cl. C12Q 1/70; A61K 45/06; A61K 38/21

(52) U.S. Cl. CPC C12Q 1/707; A61K 38/21; A61K 45/06
USPC 424/85.4; 435/5

(57) ABSTRACT

A method for predicting response of a patient infected with HCV-1a to interferon treatment.

### CROSS REFERENCE TO RELATED APPLICATION

This application is a continuation of U.S. application Ser. No. 13/036,262, filed Feb. 28, 2011, which is continuation of PCT Application Number PCT/US2009/055385, filed Aug. 28, 2009 which claims priority to U.S. Provisional Application No. 61/092,503, filed on Aug. 28, 2008. The contents of which are incorporated herein by reference in their entirety.

### FIELD OF THE INVENTION

This invention relates to methods for predicting response of a patient infected with HCV-1a to a treatment regimen including interferon.

### BACKGROUND OF THE INVENTION

Infection by hepatitis C virus (“HCV”) is a compelling human medical problem. HCV is recognized as the causative agent for most cases of non-A, non-B hepatitis, with an estimated human sero-prevalence of 3% globally (A. Alberti et al., “Natural History of Hepatitis C,” J. Hepatology, 31 (Suppl. 1), pp. 17-24 (1999)). Nearly four million individuals may be infected in the United States alone. (M. J. Alter et al., “The Epidemiology of Viral Hepatitis in the United States,” Gastroenterol. Clin. North Am., 23, pp. 437-455 (1994); M. J. Alter “Hepatitis C Virus Infection in the United States,” J. Hepatology, 31 (Suppl. 1), pp. 88-91 (1999)). Prior to the introduction of anti-HCV screening in mid-1990's, HCV accounted for 80-90% of post-transfusion hepatitis cases in the United States. A high rate of HCV infection is also seen in individuals with bleeding disorders or chronic renal failure groups that have frequent exposure to blood and blood products.

Upon first exposure to HCV, only about 20% of infected individuals develop acute clinical hepatitis while others appear to resolve the infection spontaneously. In almost 70% of instances, however, the virus establishes a chronic infection that may persist for decades. (S. Iwarson, “The Natural Course of Chronic Hepatitis,” FEMS Microbiology Reviews, 14, pp. 201-204 (1994); D. Lavanchy, “Global Surveillance and

Control of Hepatitis C,” J. Viral Hepatitis, 6, pp. 35-47 (1999)). Prolonged chronic infection can result in recurrent and progressively worsening liver inflammation, which often leads to more severe disease states such as cirrhosis and hepatocellular carcinoma. (M. C. Kew, “Hepatitis C and Hepatocellular Carcinoma”, FEMS Microbiology Reviews, 14, pp. 211-220 (1994); I. Saito et. al., “Hepatitis C Virus Infection is Associated with the Development of Hepatocellular Carcinoma,” Proc. Natl. Acad. Sci. USA, 87, pp. 6547-6549 (1990)).

HCV is an enveloped virus containing a positive-sense single-stranded RNA genome of approximately 9.5 kb. On the basis of its genome organization and virion properties, HCV has been classified as a separate genus in the family Flaviviridae, a family that also includes pestiviruses and flaviviruses (Alter, 1995, Semin. Liver Dis. 15:5-14). The viral genome consists of a lengthy 5′ untranslated region (UTR), a long open reading frame encoding a polyprotein precursor of approximately 3011 amino acids, and a short 3′ UTR. The polyprotein precursor is cleaved by both host and viral proteases to yield mature viral structural and nonstructural proteins. HCV encodes two proteinases, a zinc-dependent metalloproteinase, encoded by the NS2-NS3 region, and a serine proteinase encoded in the NS3/NS4 region. These proteinases are required for cleavage of specific regions of the precursor polyprotein into mature peptides. The carboxyl half of nonstructural protein 5B, NS5B, contains the RNA-dependent RNA polymerase. The exact function in viral replication of the remaining nonstructural proteins, NS4B, and NS5A remains unknown.

Interferon-alpha (interferon) is a Food and Drug Administration-approved treatment for chronic HCV infection. The effects of interferon are mediated through different cellular inducible proteins, including double-stranded RNA-activated protein kinase (PKR) (Gale et al., 1997, Virology 230:217-227). Only 8 to 12% of patients with HCV genotype 1 have a sustained clinical virological response (SVR) to interferon therapy (Carithers et al., 1997, Hepatology 26:83S-88S; Lindsay, 1997, Heptatology 26:71S-77S). The combination therapy of interferon with the guanosine analogue, ribavirin (RBV), was shown to be superior to interferon monotherapy in producing sustained biochemical and virological responses (Poynard et al., 1998, Lancet 352:1426-1432). However, despite the significant improvement in rates of sustained response, as many as 60% of patients with high-titer HCV genotype 1 infection are nonresponsive to pegylated-interferon and ribavirin therapy. For example, the response rate in patients infected with HCV-1 is less than 40%. Similar low response rates for patients from the United States infected with prototype genotype 1a, have also been reported (Mahaney et al. 1994, Hepatology 20:1405-1411). In contrast, the response rate of patients infected with HCV genotype-2 is nearly 80% (Fried et al., 1995, Semin. Liver Dis. 15:82-91.) Expression of the entire HCV polyprotein has been shown to inhibit interferon-induced signaling in human U2-OS osteosarcoma cells (Heim et al., 1999, J. Virol. 73:8469-8475). It was not reported which HCV protein was responsible for this effect.

The relationship between interferon-response and the nonstructural 5A (NS5A) sequence of HCV is controversial. Response to interferon therapy differs among the HCV subtypes, with the HCV-1b subtype being particularly resistant to interferon treatment (Alter et al., 1998, MMWR Recomm. Rep. 47 (RR-19):1-39). A comparison of the full length HCV nucleic acid sequence from interferon-resistant and interferon-sensitive viruses from HCV infected patients revealed missense substitutions corresponding to the carboxy terminus of the NS5A protein (Enomoto et al., 1995, J. Clin. Invest. 96:224-230). The corresponding 40 amino acid region of NS5A (amino acids 2209-2248 of the HCV polyprotein) has been termed the interferon sensitivity determining region, or ISDR (Enomoto et al., 1995). The ISDR is enclosed within a region in the NS5A protein which has been shown to bind to and inhibit the function of PKR in vitro (Gale et al., Mol. Cell Biol., 1998, 18:5208-5218). Enomoto et al. (1996, N. Eng. J. Med. 334:77-81) proposed a model in which patients who respond to interferon-therapy have viruses with multiple substitutions in the ISDR (compared to the interferon-resistant HCV 1b-J prototype sequence) whereas patients who fail interferon-therapy have viruses with few substitutions in the ISDR.

Of the studies that have published ISDR sequences from interferon-resistant and interferon-sensitive viruses, nine support the Enomoto model and conclude that, at the 5% significance level, the data provide sufficient evidence that interferon-response and substitutions in the ISDR are dependent (Enomoto et al.,1995, 1996; Chayama et al., 1997, Hepatology, 25:745-749; Kurosaki et al., 1997, Hepatology 25:750-753; Fukuda et al., 1998, J. Gastroenterol. Hepatol. 13:412-418; Saiz et al., 1998, J. Infect. Dis. 177:839-847; Murashima et al., 1999, Scand. J. Infect. Dis. 31:27-32; Sarrazin et al. 1999, J. Hepatol. 30:1004-1013; Sakuma et al., 1999, J. Infect. Dis. 180:1001-1009). The other 16 studies were unable to conclude that there is a correlation (Hofgartner et al., 1997, J. Med. Virol. 53:118-126; Khorsi et al., 1997, J. Hepatol. 27:72-77; Squadrito et al., 1997, Gastroenterology 113:567-572; Zeuzem et al., 1997, Hepatology 25:740-744; Duverlie et al., 1998, J. Gen. Virol. 79:1373-1381; Franguel et al., 1998, Hepatology 28:1674-1679; Odeberg et al., 1998, J. Med. Virol. 56:33-38; Pawlotsky et al., 1998, J. Virol. 72:2795-2805; Polyak et al., 1998, J. Virol. 72:4288-4296; Rispeter et al., 1998, J. Hepatol. 29:352-361; Chung et al., 1999, J. Med. Virol. 58:353-358; Sarrazin et al. 1999, J. Hepatol. 30:1004-1013; Squadrito et al., 1999, J. Hepatol. 30:1023-1027; Ibarrola et al., 1999, Am. J. Gastroenterol. 94:2487-2495; Mihm et al., 1999, J. Med. Virol. 58:227-234; Arase et al., 1999, Intern. Med. 38:461-466). Interestingly, seven of the nine studies that support a correlation are based on HCV isolates from Japan whereas 15 of the 16 studies that do not support a correlation are based on isolates from European and North American isolates. Although a statistically significant correlation between interferon response and ISDR sequence in North American and European studies are generally not found, there is evidence that a relationship does exist. When the intermediate and mutant classes of ISDR sequences from an individual study are combined, the response rates to interferon are higher than those in patients with the wild-type class of ISDR sequence (Herion and Hoolhagle, 1997, Hepatology 25:769-771).

### SUMMARY OF INVENTION

The present invention is based on the discovery that in human subjects infected with the HCV-1a subtype, there is a significant association between the viral NS5A sequence which evolved in the subject and his or her ultimate response to a treatment regimen containing interferon.

In one aspect of the present invention, the invention comprises a method treating a patient infected with HCV-1a with interferon-based treatment. The method includes steps of:
a) analyzing a partial or complete HCV NS5A gene of the patient; and
b) determining a criteria for predicting the likelihood of a positive response to the interferon-based treatment, wherein the criteria comprises one or more of the following elements:
   i) the number of changes in the interferon sensitivity determining region (ISDR) of the patient's HCV NS5A amino acid sequence when compared to a standard NS5A amino acid sequence; and
   ii) the sequence of amino acid residue at position 226 of the patient's HCV NS5A amino acid sequence

Specifically, patients containing a virus with high variability (e.g., 3 or more changes from consensus) in the ISDR and/or a methionine (M) or glutamic acid (E) at position 226 of the NS5A amino acid will have a high likelihood of achieving a rapid virological response (RVR) to pegylated-interferon & ribavirin therapy. For example, the ability of IFN/RBV therapy to diminish the virus below current detection limits (10 IU/ml) in 4 weeks of therapy (RVR) is highly predictive of achievement of a sustained virologic response (SVR). Conversely, patients infected with virus which do not have ISDR changes or other amino acids at position 226 of the NS5A amino acid would have less likelihood of achieving a RVR.

In certain embodiments, the method further includes a step of assigning weighting parameters for all the elements of the criteria under b) based on a sequence analysis of a population of HCV-1a infected patients and their respective response to the interferon-based treatment.

In certain embodiment, the sequence of amino acid residue at position 226 of the patient's HCV NS5A amino acid sequence is A, L, V, E or M. In one embodiment, the sequence of amino acid residue at position 226 of the patient's HCV NS5A amino acid sequence is A. In one embodiment, the sequence of amino acid residue at position 226 of the patient's HCV NS5A amino acid sequence is L. In one embodiment, the sequence of amino acid residue at position 226 of the patient's HCV NS5A amino acid sequence is E. In one embodiment, the sequence of amino acid residue at position 226 of the patient's HCV NS5A amino acid sequence is M. In one embodiment, the sequence of amino acid residue at position 226 of the patient's HCV NS5A amino acid sequence is V.

In certain embodiments, the criteria further includes an element of the sequence of amino acid residue at position 311 of the patient's HCV NS5A amino acid sequence. The criteria comprises one or more of the three elements. In certain embodiments, the method further includes a step of assigning weighting parameters to the element of the sequence of amino acid residue at position 311 of the patient's HCV NS5A amino acid sequence based on the sequence analysis of the population of HCV-1a infected patients and their respective response to the interferon-based treatment.

Again, in addition to having a certain number of changes in the ISDR and methionine (M) or glutamic acid (E) at position 226 of the NS5A amino acid sequence, if a patient infected with a virus which contains Q, R or A at position 311 of the NS5A amino acid sequence, the patient have a high likelihood of achieving a rapid virological response (RVR) to pegylated-interferon & ribavirin therapy. Conversely, patients infected with virus which does not contain Q, R or A at position 311 of the NS5A amino acid sequence will have an increased likelihood of virologic non-response to interferon-based treatment.

In an embodiment, the sequence of amino acid residue at position 311 of the patient's HCV NS5A amino acid sequence is S, P, Q, R or A. In certain embodiments, the sequence of amino acid residue at position 311 of the patient's HCV NS5A amino acid sequence is S. In certain embodiments, the sequence of amino acid residue at position 311 of the patient's HCV NS5A amino acid sequence is P. In certain embodiments, the sequence of amino acid residue at position 311 of the patient's HCV NS5A amino acid sequence is Q. In certain embodiments, the sequence of amino acid residue at position 311 of the patient's HCV NS5A amino acid sequence is R. In certain embodiments, the sequence of amino acid residue at position 311 of the patient's HCV NS5A amino acid sequence is A.

In certain embodiments, the standard NS5A amino acid sequence is H77.

In certain embodiments, the method further includes a step of analyzing a genetic polymorphism of the patient. In one embodiment, the genetic polymorphism of the patient is rs12979860.

In certain embodiments, the step of analyzing a partial or complete HCV NS5A gene of the patient includes a step of amplifying a portion of the partial or complete HCV NS5A gene using a polymerase chain reaction machine.

In certain embodiments, the method further comprises a step of determining whether the patient responses positively to the interferon-based treatment.

In certain embodiments, the method further comprises a step of administering the patient the interferon-based treatment if the patient is determined to be responsive to the interferon-based treatment.

In another aspect of the present invention, the present invention provides Use of interferon for the preparation of a medicament for the treatment of a patient infected with HCV-1a according a criteria for predicting the likelihood of a positive response to the interferon-based treatment, wherein the criteria comprises one or more of the following elements:
a) the amino acid position 226 of the HCV NS5A amino acid sequence of the patient; and
b) the number of changes in the interferon sensitivity determining region in the NS5A amino acid sequence of the patient when compared to a standard NS5A amino acid sequence.

In one embodiment, the elements of the criteria are assigned weighting parameters based on a sequence analysis of a population of HCV-1a infected patients and their respective response to the interferon-based treatment.

In one embodiment, the amino acid position 226 of the NS5A amino acid sequence is A, L, V, M or E. In one embodiment, the amino acid position 226 of the NS5A amino acid sequence is A. In one embodiment, the amino acid position 226 of the NS5A amino acid sequence is L. In one embodiment, the amino acid position 226 of the NS5A amino acid sequence is E. In one embodiment, the amino acid position 226 of the NS5A amino acid sequence is M. In one embodiment, the sequence of amino acid residue at position 226 of the patient's HCV NS5A amino acid sequence is V.

In certain embodiments, the criteria further includes an element of the amino acid residue at position 311 of the NS5A amino acid sequence of the patient, wherein the criteria comprises one or more of the three elements. In certain embodiment, the elements of the criteria are assigned weighting parameters based on a sequence analysis of the population of HCV-1a infected patients and their respective response to the interferon-based treatment.

In certain embodiment, the amino acid residue at position 311 of the NS5A amino acid sequence of the patient is S, P, Q, R or A. In one embodiment, the amino acid residue at position 311 is S. In one embodiment, the amino acid residue at position 311 is P. In one embodiment, the amino acid residue at position 311 is Q. In one embodiment, the amino acid residue at position 311 is R. In one embodiment, the amino acid residue at position 311 is A.

In one embodiment, the medicament includes one or more anti-HCV agents.

In one embodiment, the medicament includes ribavirin, a HCV protease inhibitor and a HCV polymerase inhibitor. In one embodiment, the HCV protease inhibitor is BMS-790052, MK 7009, BI 201335, SCH900518, VX-985, SCHSO3034, VX-950, R7227, ITMN-191, ACH-1095 or TMC435350. In another embodiment, the HCV protease inhibitor is VX-950. In yet another embodiment, the HCV protease inhibitor is SCHSO303. In another embodiment, the HCV polymerase inhibitor is VCH-916, IDX-184, VX-222, filibuvir, ABT-033, ABT-072, GS190, ANA598, MK-3281, BMS-650032, or R7128.

In one embodiment, the medicament further includes a NS4A inhibitor, a NS4B inhibitor, Cyclophilin inhibitor and a combination thereof. An example of the NS4A inhibitor, NS4B and Cyclophilin inhibitor is ACH-806; Clemizole; and Debio-025 and NIM811, respectively.

In one embodiment, the interferon-based treatment is selected from the group consisting of Roferon®-A, Pegasys®, Intron®, and Peg-Intron.

In one embodiment, the standard NS5A amino acid sequence is H77.

In certain embodiments, the criteria further includes a genetic polymorphism of the patient. In one embodiment, the genetic polymorphism of the patient is rs12979860.

In yet another aspect of the present invention, the present invention provides a method of prescribing a therapy regimen and/or duration for a patient infected with HCV-1a. The method comprises steps of
a) analyzing a partial or complete HCV NS5A gene of the patient; and
b) determining a criteria for predicting the likelihood of a positive response to an interferon-based treatment, wherein the criteria comprises one or more of the following elements:
   i) the number of changes in the interferon sensitivity determining region of the patient's HCV NS5A amino acid sequence when compared to a standard NS5A amino acid sequence; and
   ii) the sequence of amino acid residue at position 226 of the patient's HCV NS5A amino acid sequence; and
c) determining the therapy regimen and/or duration of the patient.

In certain embodiments, the method further includes assigning weighting parameters for all the elements of the criteria under b) based on a sequence analysis of a population of HCV-1a infected patients and their respective response to the interferon-based treatment.

In certain embodiments, the sequence of amino acid residue at position 226 of the patient's HCV NS5A amino acid sequence is A, L, V, E or M. In one embodiment, the sequence of amino acid residue at position 226 of the patient's HCV NS5A amino acid sequence is A. In one embodiment, the sequence of amino acid residue at position 226 of the patient's HCV NS5A amino acid sequence is L. In one embodiment, the sequence of amino acid residue at position 226 of the patient's HCV NS5A amino acid sequence is E. In one embodiment, the sequence of amino acid residue at position 226 of the patient's HCV NS5A amino acid sequence is M. In one embodiment, the sequence of amino acid residue at position 226 of the patient's HCV NS5A amino acid sequence is V.

In certain embodiments, the method includes the criteria that further include an element of the sequence of amino acid residue at position 311 of the patient's HCV NS5A amino acid sequence. The criteria comprise one or more of the three elements. In certain embodiment, the method includes a step of assigning a weighting parameter to the element of the sequence of amino acid residue at position 311 of the patient's HCV NS5A amino acid sequence based on a sequence analysis of a population of HCV-1a infected patients and their respective response to the interferon-based treatment.

In certain embodiments, the sequence of amino acid residue at position 311 of the patient's HCV NS5A amino acid sequence is S, P, Q, R or A. In one embodiment, the sequence of amino acid residue at position 311 of the patient's HCV NS5A amino acid sequence is S. In one embodiment, the sequence of amino acid residue at position 311 of the patient's HCV NS5A amino acid sequence is P. In one embodiment, the sequence of amino acid residue at position 311 of the patient's HCV NS5A amino acid sequence is Q. In one embodiment, the sequence of amino acid residue at position 311 of the patient's HCV NS5A amino acid sequence is R. In one embodiment, the sequence of amino acid residue at position 311 of the patient's HCV NS5A amino acid sequence is A.

In certain embodiments, the standard NS5A amino acid sequence is H77.

In certain embodiments, the step of determining a regimen and/or duration of the patient's therapy comprises administering the patient a HCV-protease inhibitor, a second STAT-C, interferon, ribavirin or a combination thereof. In one embodiment, the step of administering the patient comprises administering the patient interferon and ribavirin for a 12-week, 36-week or 48-week duration. In one embodiment, the step of administering the patient comprises administering the patient for a 12-week duration. In one embodiment, the step of administering the patient comprises administering the patient a 36-week duration. In one embodiment, the step of administering the patient comprises administering the patient for a 48-week duration.

In certain embodiments, the HCV protease inhibitor is SCHSO3034, VX-950, R7227, ITMN-191, ACH-1095 or TMC435350. In one embodiment, the HCV protease inhibitor is SCHSO3034. In one embodiment, the HCV protease inhibitor is VX-950.

In certain embodiments, the second STAT-C is a HCV polymerase inhibitor, a NS4A inhibitor, a NS4B inhibitor or Cyclophilin inhibitor. In some embodiments, the second STAT-C is VCH-916, IDX-184, VX-222, filibuvir, ABT-033, ABT-072, GS190, ANA598, MK-3281, BMS-650032, ACH-806, Clemizole, Debio-025, NIM811 or R7128.

In certain embodiments, the method further includes a step of analyzing a genetic polymorphism of the patient. In one embodiment, the genetic polymorphism is rs12979860.

In certain embodiments, the step of analyzing a partial or complete HCV NS5A gene of the patient includes a step of amplifying a portion of the partial or complete HCV NS5A gene using a polymerase chain reaction machine.

In certain embodiments, the method further comprises a step of determining whether the patient responses positively to the interferon-based treatment.

In certain embodiments, the method further comprises a step of administering the patient the interferon-based treatment if the patient is determined to be responsive to the interferon-based treatment.

### BRIEF DESCRIPTION OF THE DRAWINGS

FIG. 1 is a plot showing levels of Peg-IFN and RBV response by the subjects. Subjects whose viral RNA load was below the limit of detection (LOD) by week 4 were termed Rapid viral responders (RVR) while those whose RNA load dropped below the LOD by week 12 were termed complete early viral responders (cEVR). Partial early viral responders (pEVR) had at least a 2-log decrease in RNA load by week 12 and non-responders (NR) had less than a 2-log decrease in RNA load during the study. Trend lines for each patient group depict the means at each timepoint±standard deviation.

FIG. 2 is a plot showing the NS5A amino acid alignment shredded into 41 overlapping stretches of 40 amino acids. The first window spanned from amino acids 6 through 45; the second from amino acids 16 through 55; the third from 26 through 65, etc. Logisitic regressions were used to determine if IFN sensitivity (i.e., patient outcome group, scored ordinally) is a function of genetic variation within any of these windows (α=0.05, with Boneferroni procedure used to control Type I error). p-values (Significance level) resulting from logisitic regressions are plotted against the length of the NS5A amino acid, with the significance level of each window plotted against its mean residue (e.g., the p-value of 0.6934 for the window spanning from residues 6 through 45 is plotted at residue 26). The 40-residue stretch which has been suggested to be the ‘interferon sensitivity-determining region’ (ISDR; AA 236-275) is boxed in grey. The point corresponding to the center of this window (p=0.0003) is the only region wherein IFN sensitivity is a function of the number of mutations, significant with a Bonferroni-corrected a of 0.05.

FIG. 3 is a plot of the number of ISDR mutations in each of the response categories. The ISDR of infectious virions within rapid viral responders (RVR) is significantly enriched in mutations relative to each other outcome group while no other outcome group is significantly different from any other, as determined by 6 independent Mann-Whitney U-test pairwise comparisons (significantly different groups are indicted by ‘a’ and ‘b’ at the top of the graph). Although non-parametric comparisons based on rank-sums were employed for statistical tests, means diamonds for each group are depicted, displaying the 95% confidence interval for the mean in their height and the relative sample size in their width. Light grey bar represents the global mean for the dataset.

FIG. 4 shows pie graphs depicting the composition of virological outcome groups for 0, 1, 2, and 3 or more mutations within the ISDR, with each chart labeled below the graph. The sample size for each group is indicated above the chart and is represented in the relative area of each graph. The legend is boxed to the left of the charts. (RVR, rapid virological response; cEVR, complete early virological response; pEVR, partial early virological response; NR, non-responsive)

FIG. 5 shows the nucleic acid (FIG. 5a) and amino acid (FIG. 5b) sequences of H77.

FIG. 6 shows the nucleic acid sequences the subjects representing the varying degree of response to the interferon-based treatment: NS5-P1-6002-B0 (RVR) (FIG. 6A), NS5-P1-16004-B0 (RVR) (FIG. 6B, NS5-P1-6005-B0 (cEVR) (FIG. 6C), NS5-P1-101001-B0 (pEVR) (FIG. 6D, NS5-P1-15001-B0 (NR) (FIG. 6E).

FIG. 7 shows the amino acid sequences of the subjects of FIG. 6: NS5-P1-6002-B0 (RVR) (FIG. 7A), NS5-P1-16004-B0 (RVR) (FIG. 7B), NS5-P1-6005-B0 (cEVR) (FIG. 7C), NS5-P1-11001-B0 (pEVR) (FIG. 7D, NS5-P1-15001-B0 (NR) (FIG. 7E).

### DETAILED DESCRIPTION OF THE INVENTION

The impact of viral sequence diversity in the full-length NS5A amino acid to interferon and RBV in genotype 1a patients from the United States was investigated.

The NS5A amino acid has been implicated to affect IFN response through the induction of quasispecies (reviewed in Macdonald 2004, Tan 2001, Hofmann 2004). While the exact role of NS5A in the HCV life cycle is unknown, it has been demonstrated that NS5A is a critical part of a multi-protein complex that catalyzes the replication of the HCV genome (Egger 2002). Independent of its direct role in HCV replication, NS5A is also able to bind to numerous cellular signaling molecules which may in turn affect the modulation of cell growth and inhibit cellular apoptotic response (Macdonald, review 2004).

Additionally, NS5A has been demonstrated to bind to the interferon-induced double-stranded RNA (dsRNA)-activated protein kinase, PKR (Gale 1997). PKR is activated by binding to dsRNA. Once activated, PKR is known to phosphorylate alpha subunit 2 of the protein synthesis initiation factor 2 (eIF-2α), leading to repression of viral protein translation (Macdonald 2004). By binding to eIF-2α, NS5A interferes with the dimerization and autophosphorylation of PKR, thus inhibiting the IFN-induced host viral response pathway (Gale 1997).

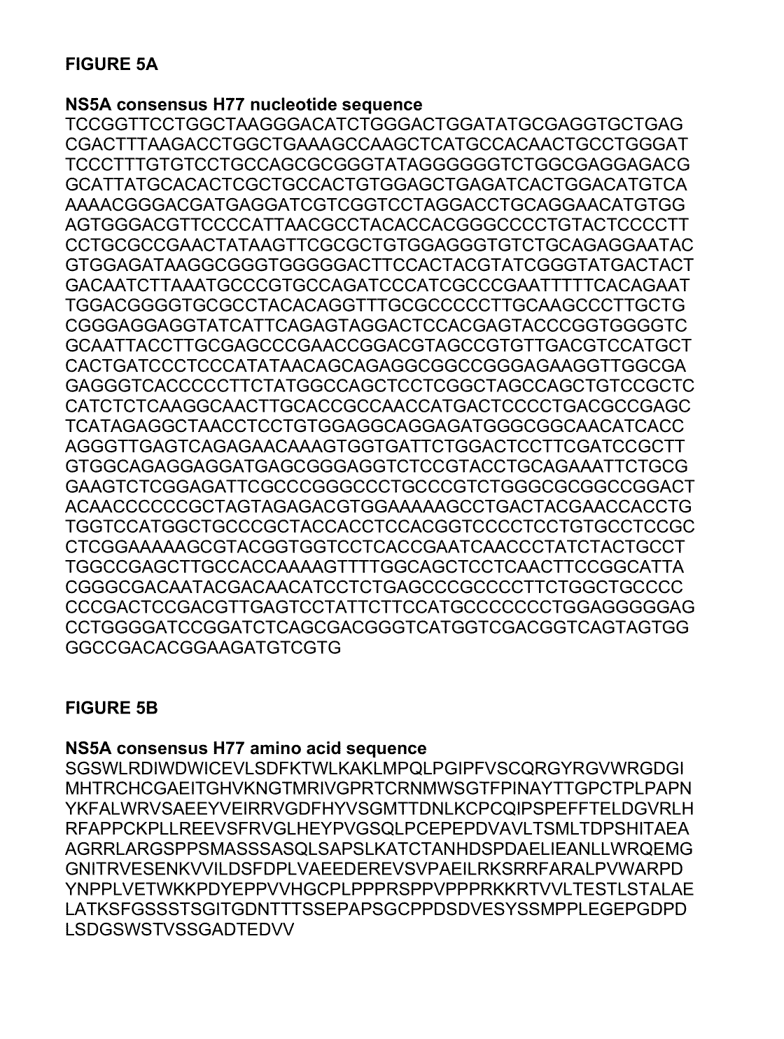

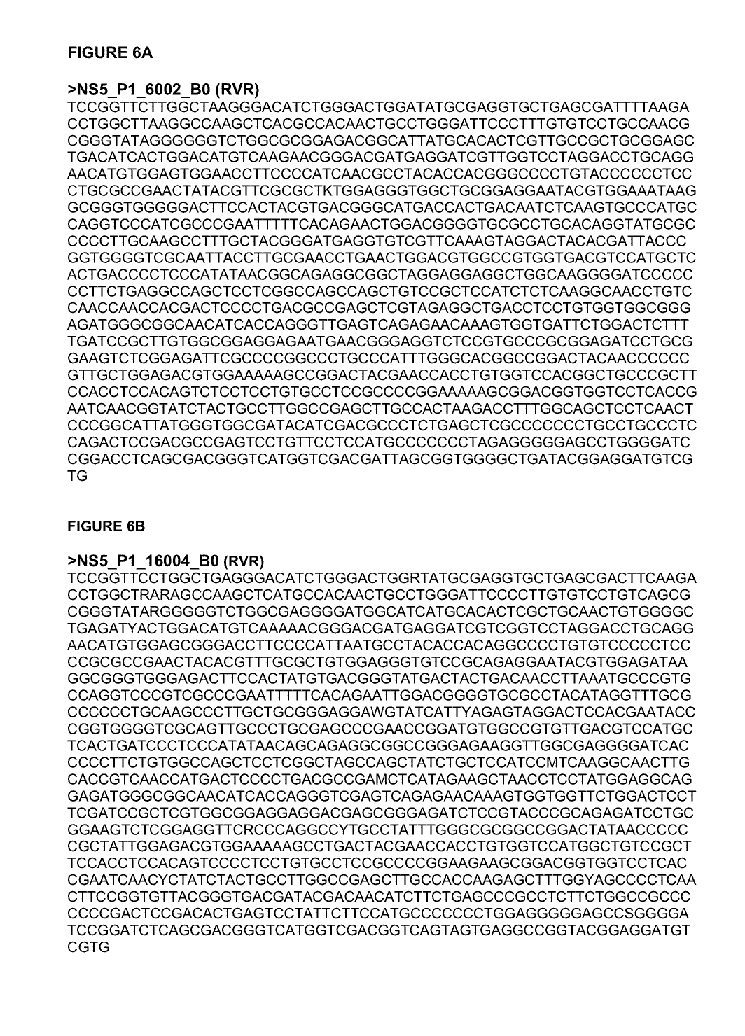

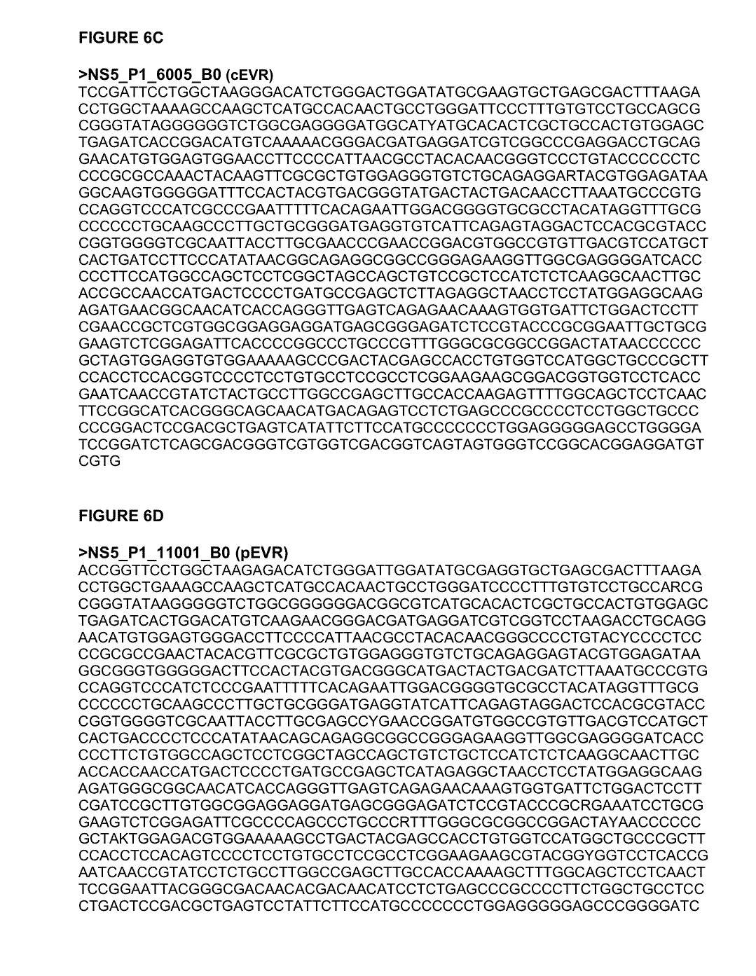

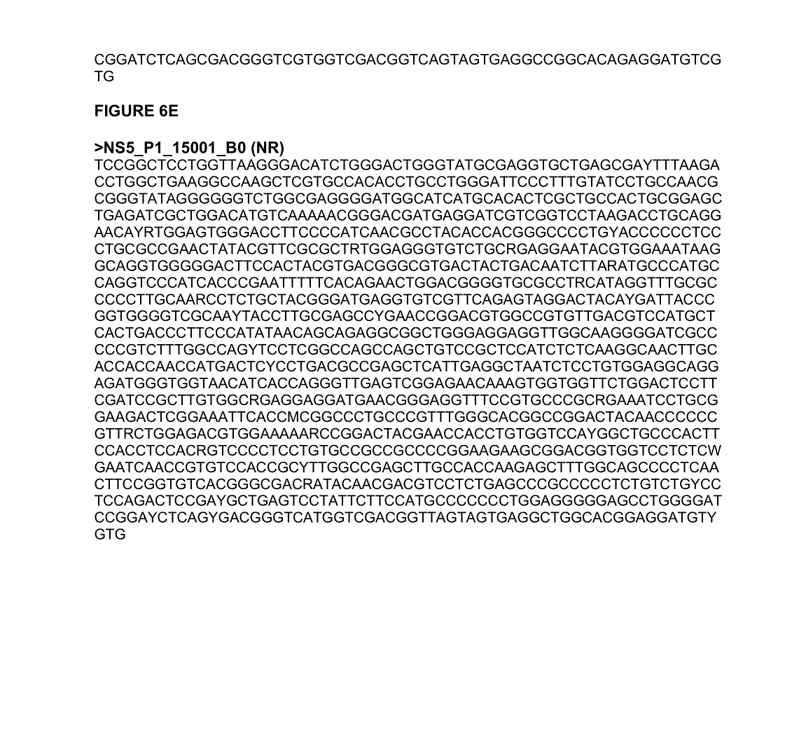

The phrase “nucleotide at position 676, 677 and 678 of the NS5A gene” means the locus at nucleotide position 676, 677 and 678 of the HCV-1a NS5A cDNA or RNA with the sequence shown in FIGS. 5 and 6 as a reference sequence for alignment, wherein the sequence shown in FIGS. 5 and 6 represents the NS5A encoding region between nucleotide position 675 and nucleotide position 679 of the HCV-1a genome nucleotide sequence.

The phrase “amino acid at position 226 of the NS5A protein” means the amino acid at position 226 of the HCV-1a NS5A protein with the sequence shown in the sequence shown in FIGS. 5 and 6 as a reference sequence for alignment wherein the sequence shown in FIGS. 5 and 6 represents the polypeptide sequence of the NS5A protein which spans from amino acid position 225 to amino acid position 227 of the HCV-1a genome polyprotein.

The phrase “nucleotide at position 931, 932, and 933 of the NS5A gene” means the locus at nucleotide position 931, 932, and 933 of the HCV-1a NS5A cDNA or RNA with the sequence shown in the sequence shown in FIGS. 5 and 6 as a reference sequence for alignment, wherein the sequence shown in FIGS. 5 and 6:1 represents the NS5A encoding region between nucleotide position 930 and nucleotide position 934 of the HCV-1a genome nucleotide.

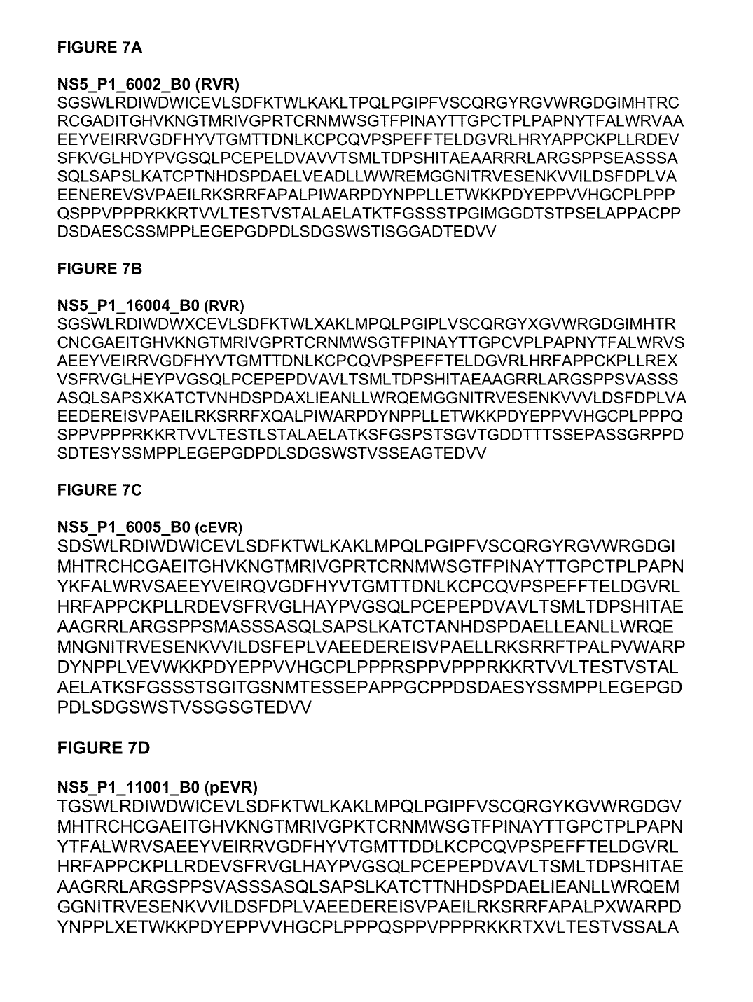

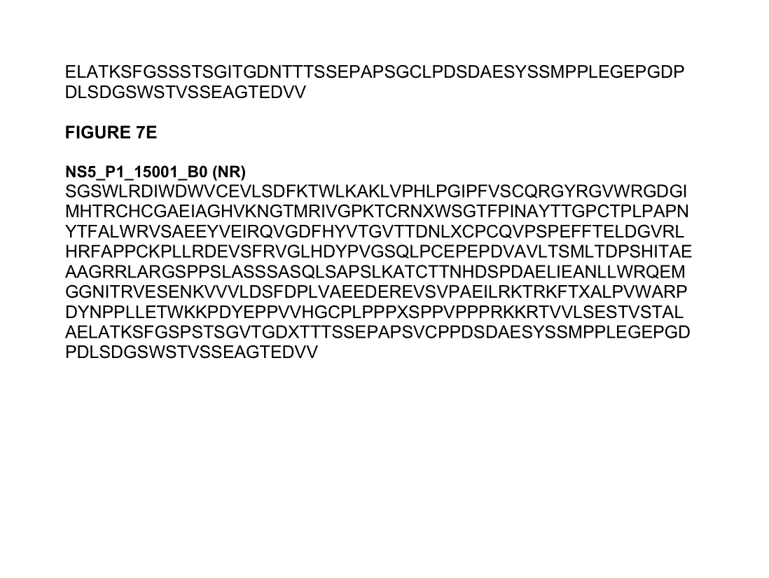

The phrase “amino acid at position 311 of the NS5A protein” means the amino acid at position 311 of the HCV-1a NS5A protein with the sequence shown in FIGS. 5 and 7 as a reference sequence for alignment wherein FIGS. 5 and 7 represents the polypeptide sequence of the NS5A protein which spans from amino acid position 310 to amino acid position 312 of the HCV-1a genome polyprotein.

The phrase “ISDR” means: (1) the nucleotide sequence between positions 705 and 826 of the HCV-1a NS5A cDNA or RNA with the sequence shown in FIGS. 5, 6 and 7 as a reference sequence for alignment; and (2) the amino acid sequence between positions 235 and 276 of the HCV-1a NS5A protein with the sequence shown in FIGS. 5, 6 and 7. However, the positions in the amino acid sequence for the ISDR may change slightly.

The terms “nucleotide substitution(s)” and “nucleotide variation(s) are herein used interchangeably and refer to nucleotide change(s) at a position in a reference nucleotide sequence of a particular gene.

The terms “amino acid mutation” and “amino acid substitution” are herein used interchangeably to refer to an amino acid change at a position in a reference protein sequence which results from a nucleotide substitution or variation in the reference nucleotide sequence encoding the reference protein.

The term “genotyping” means determining the nucleotide(s) at a particular gene locus.

The term “interferon-based treatment” refers a HCV treatment that includes administration of interferon.

The term “response” to treatment with interferon is a desirable response to the administration of an agent.

The terms “Sustained Virologic Response” (SVR) and “Complete Response” to treatment with interferon are herein used interchangeably and refer to the absence of detectable HCV RNA in the sample of an infected subject by RT-PCR both at the end of treatment and twenty-four weeks after the end of treatment. Alternatively, “sustained viral response” or “SVR” means that after dosing is completed, viral RNA levels remain undetectable. “SVR12” means that 12 weeks after dosing is completed, viral RNA levels remain undetectable. “SVR24” means that 24 weeks after dosing is completed, viral RNA levels remain undetectable.

The terms “Complete Early Virologic Response” (cEVR) is defined as at least a 99 percent (>2 log 10) reduction in HCV load (number of HCV particles in the blood) at week 12 of therapy.

The terms “Rapid Virologic Response” (RVR) used herein is defined as undetectable HCV in the blood after week 4 of therapy. 90% of patients with a RVR will have an SVR, and some patients may require only 24 weeks of treatment.

Subjects with “partial early virologic response” (pEVR) have a 100-fold decline in the HCV RNA level but continue to have detectable HCV RNA. Such patients are less likely to achieve an SVR than are subjects with cEVR.

The terms “Virologic Non-Response” and “No Response” (NR) to treatment with interferon are herein used interchangeably and refer to the presence of detectable HCV RNA in the sample of an infected subject by RT-PCR and other known conventional methods throughout treatment and at the end of treatment. Alternatively, as used herein “non-responsive” includes patients who do not achieve or maintain a sustained virologic response (SVR) (undetectable HCV RNA 24 weeks after the completion of treatment) to the standard peg-IFN with RBV treatment, and patients who have had a lack of response. Lack of response is defined as a <2-log 10 decline from baseline in HCV RNA, as a failure to achieve undetectable levels of HCV virus, or as a relapse following discontinuation of treatment. As defined above, undetectable HCV RNA means that the HCV RNA is present in less than 10 IU/mL as determined by assays currently commercially available, for example, as determined by the Roche COBAS TaqMan™ HCV/HPS assay. For example, “non-responsive” includes “week 4 null responders”, “week 12 null responders”, “week 24 null responders”, “week 26 to week 48 null responders”, “partial responders”, “viral breakthrough responders” and “relapser responders” with the standard peg-IFN with RBV treatment. A “week 4 null responder” is defined by a <1-log 10 drop in HCV RNA (not having a ≧1-log 10 decrease from baseline in HCV RNA) at week 4 of the standard peg-IFN with RBV treatment. A “week 12 null responder” is defined by a <2-log 10 drop in HCV RNA at week 12 (not having achieved an early viral response (EVR), a ≧2-log 10 decrease from the baseline in HCV RNA at week 12) of the standard peg-IFN with RBV treatment. A “week 24 null responder” is defined as a subject who has had detectable HCV RNA at week 24 of the standard peg-IFN with RBV treatment. A “week 26 to week 48 null responder” is defined as a subject who had detectable HCV RNA between weeks 26 and 48 of the standard peg-IFN with RBV treatment. A “partial responder” is defined by a ≧2-log 10 drop at week 12, but detectable HCV RNA at week 24 of the standard peg-IFN with RBV treatment. A “viral breakthrough responder” is defined by detectable HCV-RNA after achieving undetectable HCV-RNA during peg-IFN with RBV treatment. Viral breakthrough is defined as i) an increase in HCV RNA of >1-log 10 compared to the lowest recorded on-treatment value or ii) an HCV RNA level of >100 IU/mL in a patient who had undetectable HCV RNA at a prior time point. Specific examples of viral breakthrough responders include patients who have viral breakthroughs between week 4 and week 24. A “relapser responder” is a patient who had undetectable HCV RNA at completion of the peg-IFN with RBV (prior treatment) (generally 6 weeks or less after the last dose of medication), but relapsed during follow-up (e.g., during a 24-week post follow-up). A relapser responder may relapse following 48 weeks of peg-IFN with RBV treatment.

Typical peg-IFN and RBV treatment regimens include 12 weeks, 24 weeks, 36 weeks and 48 weeks treatments. Various types of peg-IFN are commercially available, for example, in vials as a prepared, premeasured solution or as a lyophilized (freeze-dried) powder with a separate diluent (mixing fluid). Pegylated interferon alfa-2b (Peg-Intron®) and alfa-2a (Pegasys®) are typical examples. Various types of interferon, including various dosage forms and formulation types, that can be employed in the invention are commercially available (see, e.g., specific examples of interferon described above). For example, various types of interferon are commercially available in vials as a prepared, premeasured solution or as a lyophilized (freeze-dried) powder with a separate diluent (mixing fluid). Pegylated interferon alfa-2b (Peg-Intron®) and alfa-2a (Pegasys®) vary from the other interferons by having molecules of polyethylene glycol (PEG) attached to them. The PEG is believed to cause the interferon to remain in the body longer and thus prolongs the effects of the interferon as well as its effectiveness. Pegylated interferon is generally administered by injection under the skin (subcutaneous). Pegasys® comes as an injectable solution in pre-filled syringes or in vials. The usual dose of Pegasys® is 180 μg, taken once a week. PEG-Intron® generally comes in a pre-filled pen that contains powder and sterile water; pushing down on the pen mixes them together. The dose of PEG-Intron® generally depends on weight-1.5 μg per kilogram (a range of between about 50 and about 150 μg total), taken once a week. In certain embodiments, a pegylated interferon, e.g., pegylated interferon-alpha 2a or pegylated interfero-alpha 2b, is employed in the invention. Typically, interferon can be dosed according to the dosage regimens described in its commercial product labels.

Ribavirin is typically administered orally, and tablet forms of ribavirin are currently commercially available. General standard, daily dose of ribavirin tablets (e.g., about 200 mg tablets) is about 800 mg to about 1200 mg (according to the dosage regimens described in its commercial product labels).

The term “STAT-C” is an abbreviation of specifically targeted antiviral therapy for Hepatitis C. This mode of therapy includes the medications that are targeting two enzymes required for Hepatitis C reproduction: serine protease and polymerase. Known as Hepatitis C protease and polymerase inhibitors.

The terms “sample” or “biological sample” refers to a sample of tissue or fluid isolated from an individual, including, but not limited to, for example, tissue biopsy, plasma, serum, whole blood, spinal fluid, lymph fluid, the external sections of the skin, respiratory, intestinal and genitourinary tracts, tears, saliva, milk, blood cells, tumors, organs. Also included are samples of in vitro cell culture constituents (including, but not limited to, conditioned medium resulting from the growth of cells in culture medium, putatively virally infected cells, recombinant cells, and cell components).

Interferon referred herein includes, but not limited to, α-, β-, γ-interferons and pegylated derivatized interferon-a compound. In some embodiments, the terms “interferon” and “interferon-alpha” are used herein interchangeably and refer to the family of highly homologous species-specific proteins that inhibit viral replication and cellular proliferation and modulate immune response. Typical suitable interferons include, but are not limited to, recombinant interferon alpha-2b such as Intron™ A interferon available from Schering Corporation, Kenilworth, N.J., recombinant interferon alpha-2a such as Roferon™-A interferon available from Hoffmann-La Roche, Nutley, N.J., recombinant interferon alpha-2C such as Berofor™ alpha 2 interferon available from Boehringer Ingelheim Pharmaceutical, Inc., Ridgefield, Conn., interferon alpha-n1, a purified blend of natural alpha interferons such as Sumiferon™ available from Sumitomo, Japan or as Wellferon™ interferon alpha-n1 (INS) available from the Glaxo-Wellcome Ltd., London, Great Britain, or a consensus alpha interferon such as those described in U.S. Pat. Nos. 4,897,471 and 4,695,623 (especially Examples 7, 8 or 9 thereof) and the specific product available from Amgen, Inc., Newbury Park, Calif., or interferon alpha-n3 a mixture of natural alpha interferons made by Interferon Sciences and available from the Purdue Frederick Co., Norwalk, Conn., under the Alferon Tradename. The use of interferon alpha-2a or alpha-2b is preferred.

The term “pegylated interferon alpha” as used herein means polyethylene glycol modified conjugates of interferon alpha, preferably interferon alpha-2a and alpha-2b. Typical suitable pegylated interferon alpha include, but are not limited to, Pegasys™ and Peg-Intron™.

As used herein, the terms “nucleic acid,” “nucleotide,” “polynucleotide” and “oligonucleotide” refer to primers, probes, oligomer fragments to be detected, oligomer controls and unlabeled blocking oligomers and shall be generic to polydeoxyribonucleotides (containing 2-deoxy-D-ribose), to polyribonucleotides (containing D-ribose), and to any other type of polynucleotide which is an N-glycoside of a purine or pyrimidine base, or modified purine or pyrimidine bases.

The term “changes in the interferon sensitivity determining region” refers to changes in the amino acid sequences of a NS5R gene when compared to the wild type of the NS5A, constituting an alternative form of the gene encoding NS5A. Changes may include insertions, additions, deletions, or substitutions. Nucleotide sequences are listed in the 5′ to 3′ direction.

The term “a standard NS5A amino acid sequence” refers to a representative amino acid sequence from a well characterized HCV sequence selected for optimal replication in cell culture systems. An example of a standard NS5A amino acid sequence is H77.

A nucleic acid, nucleotide, polynucleotide or oligonucleotide can comprise phosphodiester linkages or modified linkages such as phosphotriester, phosphoramidate, siloxane, carbonate, carboxymethylester, acetamidate, carbamate, thioether, bridged phosphoramidate, bridged methylene phosphonate, phosphorothioate, methylphosphonate, phosphorodithioate, bridged phosphorothioate or sulfone linkages, and combinations of such linkages.

A nucleic acid, nucleotide, polynucleotide or oligonucleotide can comprise the five biologically occurring bases (adenine, guanine, thymine, cytosine and uracil) and/or bases other than the five biologically occurring bases. For example, a polynucleotide of the invention might contain at least one modified base moiety which is selected from the group including but not limited to 5-fluorouracil, 5-bromouracil, 5-chlorouracil, 5-iodouracil, hypoxanthine, xanthine, 4-acetylcytosine, 5-(carboxyhydroxymethyl)uracil, 5-carboxymethylaminomethyl-2-thiouridine, 5-carboxymethylaminomethyluracil, dihydrouracil, beta-D-galactosylqueosine, inosine, N6-isopentenyladenine, 1-methylguanine, 1-methylinosine, 2,2-dimethylguanine, 2-methyladenine, 2-methylguanine, 3-methylcytosine, 5-methylcytosine, N6-methyladenine, 7-methylguanine, 5-methylaminomethyluracil, 5-methoxyaminomethyl-2-thiouracil, beta-D mannosylqueosine, 5′-methoxycarboxymethyluracil, 5-methoxyuracil, 2-methylthio-N6-isopentenyladenine, uracil-5-oxyacetic acid (v), wybutoxosine, pseudouracil, queosine, 2-thiocytosine, 5-methyl-2-thiouracil, 2-thiouracil, 4-thiouracil, 5-methyluracil, uracil-5-oxyacetic acidmethylester, 3-(3-amino-3-N-2-carboxypropyl) uracil, (acp3)w, 2,6-diaminopurine, and 5-propynyl pyrimidine.

Furthermore, a nucleic acid, nucleotide, polynucleotide or oligonucleotide can comprise one or more modified sugar moieties such as arabinose, 2-fluoroarabinose, xylulose, and hexose.

It is not intended that the present invention be limited by the source of a nucleic acid, nucleotide, polynucleotide or oligonucleotide. A nucleic acid, nucleotide, polynucleotide or oligonucleotide can be from a human or non-human mammal, or any other organism, or derived from any recombinant source, or synthesized in vitro or by chemical synthesis. A nucleic acid, nucleotide, polynucleotide or oligonucleotide may be DNA, RNA, cDNA, DNA-RNA, locked nucleic acid (LNA), peptide nucleic acid (PNA), a hybrid or any mixture of the same, and may exist in a double-stranded, single-stranded or partially double-stranded form. The nucleic acids of the invention include both nucleic acids and fragments thereof, in purified or unpurified forms, including genes, chromosomes, plasmids, the genomes of biological material such as microorganisms, e.g., bacteria, yeasts, viruses, viroids, molds, fungi, plants, animals, humans, and the like.

There is no intended distinction in length between the terms nucleic acid, nucleotide, polynucleotide and oligonucleotide, and these terms will be used interchangeably. These terms include double- and single-stranded DNA, as well as double- and single-stranded RNA.

“Corresponding” means identical to or complementary to a designated sequence.

Because mononucleotides can be reacted to make oligonucleotides in a manner such that the 5′ phosphate of one mononucleotide pentose ring is attached to the 3′ oxygen of its neighbor in one direction via a phosphodiester linkage, an end of an oligonucleotide is referred to as the “5′ end” if its 5′ phosphate is not linked to the 3′ oxygen of a mononucleotide pentose ring and as the “3′ end” if its 3′ oxygen is not linked to a 5′ phosphate of a subsequent mononucleotide pentose ring. As used herein, a nucleic acid sequence, even if internal to a larger oligonucleotide, also may be said to have 5′ and 3′ ends.

When two different, non-overlapping oligonucleotides anneal to different regions of the same linear complementary nucleic acid sequence, and the 3′ end of one oligonucleotide points toward the 5′ end of the other, the former may be called the “upstream” oligonucleotide and the latter the “downstream” oligonucleotide.

The term “primer” may refer to more than one primer or a mixture of primers and refers to an oligonucleotide, whether occurring naturally, as in a purified restriction digest, or produced synthetically, which is capable of acting as a point of initiation of polynucleotide synthesis along a complementary strand when placed under conditions in which synthesis of a primer extension product which is complementary to a nucleic acid strand is catalyzed. Such conditions typically include the presence of four different deoxyribonucleoside triphosphates and a polymerization-inducing agent such as DNA polymerase or reverse transcriptase, in a suitable buffer (“buffer” includes substituents which are cofactors, or which affect pH, ionic strength, etc.), and at a suitable temperature. The primer is preferably single-stranded for maximum efficiency in amplification.

rs12979860 is a polymorphism on chromosome 19, which is reported to be associated with SVR in HCV patient groups. The polymorphism resides 3 kilobases (kb) upstream of the IL28B gene, encoding IFN-λ-3. In some embodiment, the methods of the present invention for predicting response of a patient infected with HCV-1a to interferon-based treatment can be based on an analysis of:

a partial or complete HCV NS5A gene of the patient; and

the polymorphism on chromosome 19 of the patient.

The complement of a nucleic acid sequence as used herein refers to an oligonucleotide which, when aligned with the nucleic acid sequence such that the 5′ end of one sequence is paired with the 3′ end of the other, is in “antiparallel association.” Certain bases not commonly found in natural nucleic acids may be included in the nucleic acids of the present invention and include, for example, inosine, 7-deazaguanine and those discussed above. Complementarity need not be perfect; stable duplexes may contain mismatched base pairs or unmatched bases. Those skilled in the art of nucleic acid technology can determine duplex stability by empirically considering a number of variables including, for example, the length of the oligonucleotide, base composition and sequence of the oligonucleotide, ionic strength, and incidence of mismatched base pairs.

As used herein, the term “probe” refers to an oligonucleotide which can form a duplex structure with a region of a nucleic acid, due to complementarity of at least one sequence in the probe with a sequence in the region and is capable of being detected. The probe, preferably, does not contain a sequence complementary to sequence(s) of a primer in a 5′ nuclease reaction. As discussed below, the probe can be labeled or unlabeled. The 3′ terminus of the probe can be “blocked” to prohibit incorporation of the probe into a primer extension product. “Blocking” can be achieved by using non-complementary bases or by adding a chemical moiety such as biotin or a phosphate group to the 3′ hydroxyl of the last nucleotide, which may, depending upon the selected moiety, serve a dual purpose by also acting as a label for subsequent detection or capture of the nucleic acid attached to the label. Blocking can also be achieved by removing the 3′-OH or by using a nucleotide that lacks a 3′-OH such as a dideoxynucleotide.

The term “label” as used herein refers to any atom or molecule which can be used to provide a detectable (optionally quantifiable) signal, and which can be attached to a nucleic acid or protein. Labels may provide signals detectable by fluorescence, radioactivity, colorimetry, gravimetry, X-ray diffraction or absorption, magnetism, enzymatic activity, and the like. Convenient labels for the present invention include those that facilitate detection of the size of an oligonucleotide fragment.

In certain embodiments of the invention, a “label” is a fluorescent dye. Fluorescent labels may include dyes that are negatively charged, such as dyes of the fluorescein family, or dyes that are neutral in charge, such as dyes of the rhodamine family, or dyes that are positively charged, such as dyes of the cyanine family. Dyes of the fluorescein family include, e.g., FAM, HEX, TET, JOE, NAN and ZOE. Dyes of the rhodamine family include Texas Red, ROX, R110, R6G, and TAMRA. FAM, HEX, TET, JOE, NAN, ZOE, ROX, R110, R6G, and TAMRA are marketed by Perkin-Elmer (Foster City, Calif.), and Texas Red is marketed by Molecular Probes, Inc. (Eugene, Oreg.). Dyes of the cyanine family include Cy2, Cy3, Cy5, and Cy7 and are marketed by Amersham (Amersham Place, Little Chalfont, Buckinghamshire, England).

The term “quencher” as used herein refers to a chemical moiety that absorbs energy emitted from a fluorescent dye, for example, when both the quencher and fluorescent dye are linked to a common polynucleotide. A quencher may re-emit the energy absorbed from a fluorescent dye in a signal characteristic for that quencher and thus a quencher can also be a “label.” This phenomenon is generally known as fluorescent resonance energy transfer or FRET. Alternatively, a quencher may dissipate the energy absorbed from a fluorescent dye as heat. Molecules commonly used in FRET include, for example, fluorescein, FAM, JOE, rhodamine, R6G, TAMRA, ROX, DABCYL, and EDANS. Whether a fluorescent dye is a label or a quencher is defined by its excitation and emission spectra, and the fluorescent dye with which it is paired. For example, FAM is most efficiently excited by light with a wavelength of 488 nm, and emits light with a spectrum of 500 to 650 nm, and an emission maximum of 525 nm. FAM is a suitable donor label for use with, e.g., with TAMRA as a quencher which has at its excitation maximum 514 nm. Exemplary non-fluorescent quenchers that dissipate energy absorbed from a fluorescent dye include the Black Hole Quenchers™ marketed by Biosearch Technologies, Inc. (Novato, Calif).

As defined herein, “5′ to 3′ nuclease activity” refers to that activity of a template-specific nucleic acid polymerase including either a 5′ to 3′ exonuclease activity traditionally associated with some DNA polymerases whereby nucleotides are removed from the 5′ end of an oligonucleotide in a sequential manner, (e.g., E. coli DNA polymerase I has this activity whereas the Klenow fragment does not), or a 5′ to 3′ endonuclease activity wherein cleavage occurs more than one phosphodiester bond (nucleotide) from the 5′ end, or both. Although not intending to be bound by any particular theory of operation, the preferred substrate for 5′ to 3′ endonuclease activity-dependent cleavage on a probe-template hybridization complex is a displaced single-stranded nucleic acid, a fork-like structure, with hydrolysis occurring at the phosphodiester bond joining the displaced region with the base-paired portion of the strand, as discussed in Holland et al., 1991, Proc. Natl. Acad. Sci. USA 88:7276-80, hereby incorporated by reference in its entirety.

The term “adjacent” as used herein refers to the positioning of the primer with respect to the probe on its complementary strand of the template nucleic acid. The primer and probe may be separated by more than 20 nucleotides, by 1 to about 20 nucleotides, more preferably, about 1 to 10 nucleotides, or may directly abut one another, as may be desirable for detection with a polymerization-independent process. Alternatively, for use in the polymerization-dependent process, as when the present method is used in a PCR amplification and detection methods as taught herein, the “adjacency” may be anywhere within the sequence to be amplified, anywhere downstream of a primer such that primer extension will position the polymerase so that cleavage of the probe occurs.

As used herein, the term “thermostable nucleic acid polymerase” refers to an enzyme which is relatively stable to heat when compared, for example, to nucleotide polymerases from E. coli and which catalyzes the polymerization of nucleoside triphosphates. Generally, the enzyme will initiate synthesis at the 3′-end of the primer annealed to the target sequence, and will continue synthesis of a new strand toward the 5′-end of the template, and if possessing a 5′ to 3′ nuclease activity, hydrolyzing intervening, annealed probe to release both labeled and unlabeled probe fragments, until synthesis terminates or probe fragments melt off the target sequence. A representative thermostable enzyme isolated from Thermus aquaticus (Taq) is described in U.S. Pat. No. 4,889,818 and a method for using it in conventional PCR is described in Saiki et al., 1988, Science 239:487-91.

Taq DNA polymerase has a DNA synthesis-dependent, strand replacement 5′-3′ exonuclease activity. See Gelfand, “Taq DNA Polymerase” in PCR Technology Principles and Applications for DNA Amplification, Erlich, Ed., Stockton Press, N.Y. (1989), Chapter 2. In solution, there is little, if any, degradation of probes.

The term “5′ nuclease reaction” of a nucleic acid, primer and probe refers to the degradation of a probe hybridized to the nucleic acid when the primer is extended by a nucleic acid polymerase having 5′ to 3′ nuclease activity, as described in detail below. Such reactions are based on those described in U.S. Pat. Nos. 6,214,979, 5,804,375, 5,487,972 and 5,210,015, which are hereby incorporated by reference in their entireties.

The term “target nucleic acid” refers to a nucleic acid which can hybridize with a primer and probe in a 5′ nuclease reaction and contains one or more nucleotide variation sites.

The terms “stringent” or “stringent conditions”, as used herein, denote hybridization conditions of low ionic strength and high temperature, as is well known in the art. See, e.g., Sambrook et al., 2001, Molecular Cloning: A Laboratory Manual, Third Edition, Cold Spring Harbor Laboratory Press, Cold Spring Harbor, N.Y.; Current Protocols in Molecular Biology (Ausubel et al., ed., J. Wiley & Sons Inc., New York, 1988); Tijssen, 1993, “Overview of principles of hybridization and the strategy of nucleic acid assays” in Laboratory techniques in biochemistry and molecular biology: Hybridization with nucleic acid probes (Elsevier), each of which is hereby incorporated by reference. Generally, stringent conditions are selected to be about 5-30 DEG C. lower than the thermal melting point (Tm) for the specified sequence at a defined ionic strength and pH. Alternatively, stringent conditions are selected to be about 5-15 DEG C. lower than the Tm for the specified sequence at a defined ionic strength and pH. The Tm is the temperature (under defined ionic strength, pH and nudeic acid concentration) at which 50% of the probes complementary to the target hybridize to the target sequence at equilibrium (as the target sequences are present in excess, at Tm, 50% of the probes are occupied at equilibrium). For example, stringent hybridization conditions will be those in which the salt concentration is less than about 1.0 M sodium (or other salts) ion, typically about 0.01 to about 1 M sodium ion concentration at about pH 7.0 to about pH 8.3 and the temperature is at least about 25 DEG C. for short probes (e.g., 10 to 50 nucleotides) and at least about 55 DEG C. for long probes (e.g., greater than 50 nucleotides). Stringent conditions may also be modified with the addition of hybridization destabilizing agents such as formamide. An exemplary non-stringent or low stringency condition for a long probe (e.g., greater than 50 nucleotides) would comprise a buffer of 20 mM Tris, pH 8.5, 50 mM KCl, and 2 mM MgCl2, and a reaction temperature of 25 DEG C.

The practice of the present invention will employ, unless otherwise indicated, conventional techniques of molecular biology, microbiology and recombinant DNA techniques, which are within the skill of the art. Such techniques are explained fully in the literature. See, e.g., Sambrook et al., 2001, Molecular Cloning: A Laboratory Manual, Third Edition, Cold Spring Harbor Laboratory Press, Cold Spring Harbor, N.Y.; Oligonucleotide Synthesis (M. J. Gait, ed., 1984); Nucleic Acid Hybridization (B. D. Hames & S. J. Higgins, eds., 1984); A Practical Guide to Molecular Cloning (B. Perbal, 1984); and a series, Methods in Enzymology (Academic Press, Inc.).

The amino acid sequence of the entire HCV-1a genome is provided as FIGS. 5 and 7.

In one embodiment, the present invention provides an assay capable of detecting a nucleotide substitution at position 676, 677 or 678 of the NS5A gene.

In another embodiment, the present invention provides an assay capable of detecting a nucleotide substitution at position 931, 932 or 933 of the NS5A gene.

Numerous techniques for detecting nucleotide or amino acid variations are known in the art and can all be used to practice the methods of the present invention. The particular method used to identify the sequence variation is not a critical aspect of the invention. Although considerations of performance, cost, and convenience will make particular methods more desirable than others, it is desired that any method that can determined the number of variants in ISDR and identify the nucleotide at positions 676, 677, 678, 931, 932 and 933 of FIGS. 5 and 6 or the amino acid at positions 226 and/or 311 of FIGS. 5 and 7 will provide the information needed to practice the invention. The techniques can be polynucleotide-based or protein-based. In either case, the techniques used must be sufficiently sensitive so as to accurately detect single nucleotide or amino acid variations. Examples of the techniques can include, but not limited to, the following:
polynucleotide based detection methods (i.e. See U.S. Pat. Nos. 5,310,625; 5,322,770; 5,561,058; 5,641,864; and 5,693,517; see also Myers and Sigua, Myers and Sigua, Amplification of RNA: High-temperature reverse transcription and DNA amplification with Thermus thermophilus DNA polymerase. In: M. A. Innis, D. H. Gelfand and J J Sninsky, Editors, PCR Strategies, Academic Press, San Diego (1995), pp. 58-68)), DNA sequencing methods (i.e. DNA Sequencing methods by PE Biosystems (Foster City, Calif.); see Sanger et al., 1977 , Proc. Natl. Acad. Sci. 74:5463-5467);
amplification based genotyping methods (i.e. U.S. Pat. Nos. 4,683,195; 4,683,202; and 4,965,188; also PCR Applications, 1999, (Innis et al., eds., Academic Press, San Diego), PCR Strategies, 1995, (Innis et al., eds., Academic Press, San Diego); PCR Protocols, 1990, (Innis et al., eds., Academic Press, San Diego); and PCR Technology, 1989, (Erlich, ed., Stockton Press, New York);
ligase chain reaction (i.e. Wu and Wallace 1988, Genomics 4:560-569); the strand displacement assay (Walker et al., 1992 , Proc. Natl. Acad. Sci. USA 89:392-396, Walker et al. 1992, Nucleic Acids Res. 20:1691-1696, and U.S. Pat. No. 5,455,166); and several transcription-based amplification systems, including the methods described in U.S. Pat. Nos. 5,437,990; 5, 409,818; and 5,399,491; the transcription amplification system (TAS) (Kwoh et al., 1989, Proc. Natl. Acad. Sci. USA 86:1173-1177); and self-sustained sequence replication (3SR) (Guatelli et al., 1990, Proc. Natl. Acad. Sci. USA 87:1874-1878 and WO 92/08800);
sequence-specific amplification or primer extension methods (i.e. U.S. Pat. Nos. 5,137,806; 5,595,890; 5,639,611; and U.S. Pat. No. 4,851, 331);
Kinetic PCR-methods (i.e. Higuchi et al., 1992, Bio/Technology 10:413-417; Higuchi et al., 1993, Bio/Technology 11: 1026-1030; Higuchi and Watson, in PCR Applications, supra, Chapter 16; U.S. Pat. No. 5,994,056; and European Patent Publication Nos. 487,218 and 512,334);
Probe-based method, which rely on the difference instability of hybridization duplexes formed between the probe and the nucleotide variants, which differ in the degree of complementarity (i.e. Conner et al., 1983, Proc. Natl. Acad. Sci. USA 80:278-282, and U.S. Pat. Nos. 5,468, 613; 5,604,099; 5,310, 893; 5,451,512; 5,468,613; and 5,604,099);
Mass spectrometry (i.e. MALDI-MS; U.S. Pat. No. 6,258,539);
Protein-based detection techniques (i.e. Protein sequencing, immunoaffinity assays, enzyme-linked immunosorbent assay (ELISA); radioimmuno-assay (RIA); immunoradiometric assays (IRMA) and immunoenzymatic assays (IEMA); see e.g. U.S. Pat. Nos. 4,376,110 and 4,486,530)

In a polynucleotide-based detection method, genotyping is accomplished by identifying the nucleotide present at the substitution site, nucleotide position 931, 932 or 933 of FIGS. 5 and 6. Any type of biological sample from a HCV-1a-infected individual containing HCV-1a polynucleotide may be used for determining the genotype. Genotyping may be carried out by isolating HCV RNA using standard RNA extraction methods well known in the art. Amplification of RNA can be carried out by first reverse-transcribing the target RNA using, for example, a viral reverse transcriptase, and then amplifying the resulting cDNA, or using a combined high-temperature reverse-transcription-polymerase chain reaction (RT-PCR), as described in U.S. Pat. Nos. 5,310,652; 5,322,770; 5, 561,058; 5,641,864; and 5,693,517; each incorporated herein by reference (see also Myers and Sigua, 1995, in PCR Strategies, supra, chapter 5). A number of methods are known in the art for identifying the nucleotide present at a single nucleotide position.

The present invention also relates to kits, container units comprising useful components for practicing the present method. A useful kit can contain oligonucleotides used to detect the nucleotide substitution at positions 676, 676, 678, 931, 932 and 933 in the NS5A gene. In some cases, detection probes may be fixed to an appropriate support membrane. The kit can also contain amplification primers for amplifying a region of the NS5A locus encompassing the substitution site(s), as such primers are useful in the preferred embodiment of the invention. Alternatively, useful kits can contain a set of primers comprising a sequence-specific primer for the specific amplification of the NS5A gene. Other optional components of the kits include additional reagents used in the genotyping methods as described herein. For example, a kit additionally can contain an agent to catalyze the synthesis of primer extension products, substrate nucleoside triphosphates, means for labeling and/or detecting nucleic acid (for example, an avidin-enzyme conjugate and enzyme substrate and chromogen if the label is biotin), appropriate buffers for amplification or hybridization reactions, and instructions for carrying out the present method.

The methods disclosed herein were derived from a stepwise multivariate original logistic regression analysis.

The examples of the present invention presented below are provided only for illustrative purposes and not to limit the scope of the invention. Numerous embodiments of the invention within the scope of the claims that follow the examples will be apparent to those of ordinary skill in the art from reading the foregoing text and following examples.

### EXAMPLES

The full length NS5A protein from American patients enrolled in the control arm of clinical trial was analyzed to determine regions that may confer a positive Peg-IFN and RBV response. Eighty treatment-naïve patients received 48 weeks of Peg-IFN and RBV. Baseline viral genotypes were analyzed by population sequencing. 55 patients were infected with genotype 1a HCV. Patients were grouped by initial response to treatment i.e., rapid viral response (RVR), complete early viral response (cEVR), partial early viral.

### Example 1

### Subject Population

The study included 250 treatment-naïve subjects who had chronic, genotype 1 HCV infection. All subjects were between 18 and 65 years of age, had detectable baseline plasma HCV RNA levels, and were HBsAg and HIV antibody negative. Plasma HCV RNA levels were determined using the Roche COBAS® TaqMan HCV/HPS assay (Roche Molecular Systems Inc., Branchburg, N.J., USA). The lower limit of quantitation for the HCV RNA assay was 30 IU/mL and the limit of detection (LOD) was 10 IU/mL.

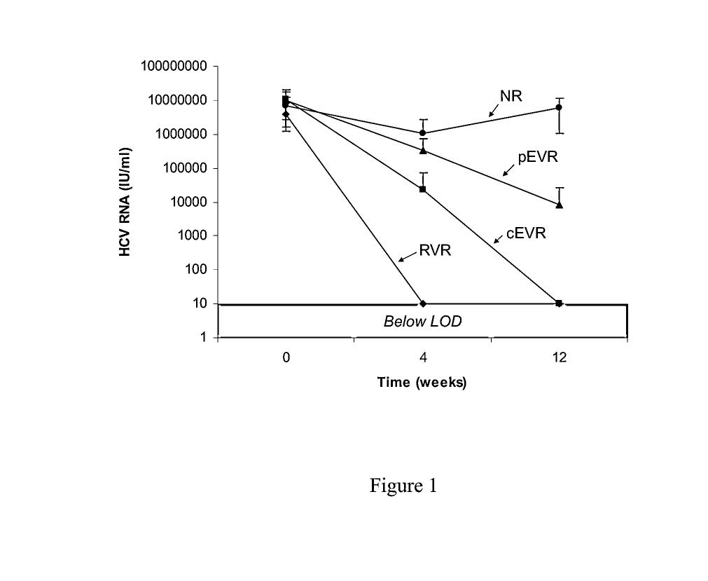

Subjects were randomized to receive TVR 750 mg q8h, peginterferon-alfa-2a (Peg-IFN) 180 μg/week, and ribavirin (RBV) 1000-1200 mg/day for 12 weeks followed by 0, 12, or 36 weeks of Peg-IFN and RBV, or TVR/Peg-IFN (no RBV) for 12 weeks. The control group received 48 weeks of Placebo/Peg-IFN and RBV. Initial treatment results were based on plasma HCV RNA levels quantified at specific intervals after the first dosing of treatment. Rapid viral responders (RVR) were classified by undetectable (<10 IU/mL) HCV RNA in plasma at week 4. Complete early viral responders (cEVR) had HCV RNA that was below the limit of detection (<10 IU/mL) at week 12. Partial early viral responders (pEVR) had a 2 log drop of HCV RNA at week 12 and non-responders (NR) had a 0 or 1 log drop of HCV RNA at week 12 (Hoefs 2007, Pealman 2007) (FIG. 1).

### Example 2

Amplification and Sequencing of HCV from Subject Plasma

Population sequence analysis of the full-length NS5A was conducted in 250 treatment-naïve subjects with genotype 1 HCV before dosing (Day 1). A 4 mL blood sample was collected from subjects by venipuncture of a forearm vein into tubes containing EDTA (K₂) anticoagulant. Plasma was separated by 10 minutes of centrifugation, aliquoted, and stored at −80° C. Sequence analysis of HCV was done by nested reverse-transcriptase polymerase chain reaction (RT-PCR) amplification of an approximately 9 kb HCV RNA fragment spanning the HCV polyprotein coding region. The DNA from this PCR was purified using the QIAquick 96 PCR Purification kit (Qiagen) and was analyzed on an agarose gel. The quality and quantity of the purified PCR product were measured by EnVision™ Multilabel Reader (PerkinElmer Waltham, Mass.). Sequencing of purified PCR product was performed by Agencourt® Biosciences (Beverly, Mass.) for using primers designed to span the entire NS5A region. The sequencing assay was successful in samples containing >1000 IU/mL of HCV RNA. The nucleic acid sequence of the subjects representing the degree of response to the interferon-based treatment, shown in FIGS. 6A-6E, was translated into the amino acid sequence, which is shown in FIGS. 7A-7E.

Sequences were aligned and analyzed using the software Mutational Surveyor (SoftGenetics, State College, Pa.).

### Example 3

### Sequence-Independent Analysis

Sequences were aligned against Hepatitis C reference genome H77 (Genbank accession: NC_{—}004102) using the default parameters of ClustalX (Gonnet Matrix, Gap opening penalty=10, Gap extension penalty=0.2 [Thompson et al., 1997]). This sequence was used as a reference in the identification of variable loci in each patient's amino acid sequence, in a comparable manner to the use of reference HCV genome D90208 in Enomoto's 1996 study. Each patients sequence was recoded into a binary matrix, with variable positions indicated by a ‘1’ and positions with the same residue as the reference being assigned a value of ‘0.’ To determine if the distribution of mutations was normally distributed across outcome groups (i.e., RVR, EVR, pEVR, and NR), a Chi-Squared test was employed at each residue of the NS5A protein. Further, the mutation frequency for each residue was calculated for each outcome group. This mutation frequency was normalized against the mutation frequency for non-responding patients (NR) to determine those residues enriched or depauperate in mutations within each outcome group.

Data were analyzed to determine if demographics (sex, race), initial viral load, or the number of mutations in numerous domains of purported functional significance within NS5A, including (i) the region responsible for cytoretention, (ii) a hyperphosphorylation domain, (iii) the interferon sensitivity determining region (ISDR), (iv) the PKR-binding domain, (v) the nuclear localization signal, and (vi) the V3 region, can be used to predict responsiveness of HCV to pegylated interferon. Each of these variables was used as an independent variable in a univariate analysis using Chi-Squared tests or logistic regression, as appropriate based on the independent variable type. All of the independent variables were also combined and used as predictors in a stepwise multiple ordinal regression mixed (forward and reverse direction) model. The alpha level required for entry into the model based on univariate statistics was set to 0.15. For all logistic regressions, each patient's response was recoded into an ordinal scale in the following order: NR (n=9), pEVR (n=11), EVR (n=26), RVR (n=9).

Outcome groups were also compared to test for a significant difference between them in terms of the number of mutations in the functional domains defined above. Where assumptions of parametric testing were met, analyses of variance with post hoc Tukey comparisons were employed with a modification to allow for unequal sample size comparisons (Kramer, 1956). Where assumptions of parametric statistics were violated, rank sums testing (Kruskal-Wallis) was employed. All statistical analyses were conducted using either SAS (v. 9.1, Sas Institute, Cary, N.C., USA) or JMP (v. 7.0, Sas Institute).

### Example 4

### Sequence-Dependent Analyses

To determine if patient sequence of domains within NS5A cluster based on their responsiveness to Peg-IFN and RBV, the NS5A amino acid alignment was divided into the previously defined domains (using Genedoc (Nichols and Nichols, 1997). A distance matrix was generated for each domain alignment using the default parameters of the program protdist (part of the Phylip package, v. 3.67 [Felsenstein, 2007]) using a Kimura substitution matrix (Kimura, 1980). Sequence clusters were generated using a nearest neighbor-joining algorithm (Saitou and Nei, 1987). Star phylogenies were assessed to determine if sequences clustered based on sequence similarity over the domain.

Additionally, to determine if mutations at specific residues within the ISDR might be responsible for imparting greater sensitivity of HCV to Peg-IFN and RBV, we regressed the character at each residue against viral responsiveness in a multivariate stepwise ordinal logistic regression. Race was included as a variable in this analysis since it had been shown to significantly affect viral response in the ‘sequence independent’ multivariate model (see Results). This analysis utilized a forward stepwise regression model; the significance level required for entry into the model based on univariate statistics was set to 0.15 while the significance level required to remain in the multivariate model was set to 0.10.

Initial treatment response for the 55 genotype la patients in the study included 7 patients that achieved RVR, 24 that achieved cEVR, 14 that achieved pEVR, and 10 that were NR. The data set comprises a 446 residue alignment of these 55 NS5A sequences, totaling 24695 amino acid positions. The majority of these residues (˜94.6%) were identical to H77, with 275 aligned positions (˜61%) invariant across the alignment. The 1338 mutations observed in our dataset were distributed amongst the remaining 174 aligned positions.

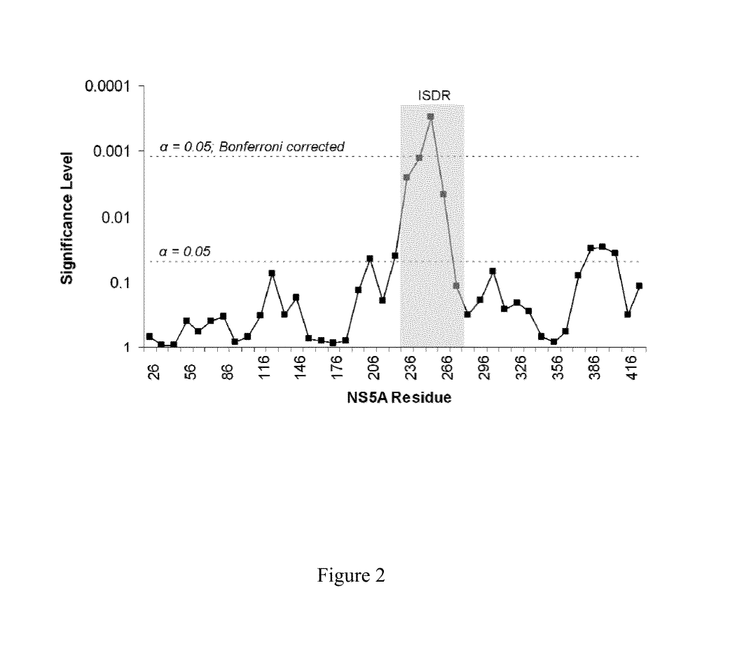

Across NS5A, there was a significant difference between outcome groups (Krukal-Wallis non-parametric one-way analysis of variance; p=0.0306), with RVR patients (median number of NS5A mutations per patient=31) having more mutations than cEVR (median=24), pEVR (median=21), and NR patients (median=26.5). Logistic regression was used to test if viral sensitivity to Peg-IFN and RBV was a function of the number of mutations within any region of the NS5A protein. Regressions were performed independently on 41 overlapping stretches of 40-amino acid residues. Peg-IFN and RBV sensitivity was found to be a function of viral heterogeneity within the ISDR (Logisitc regression; χ=13.02, p=0.0003) but was not significantly correlated with heterogeneity within any other NS5A region (FIG. 2).

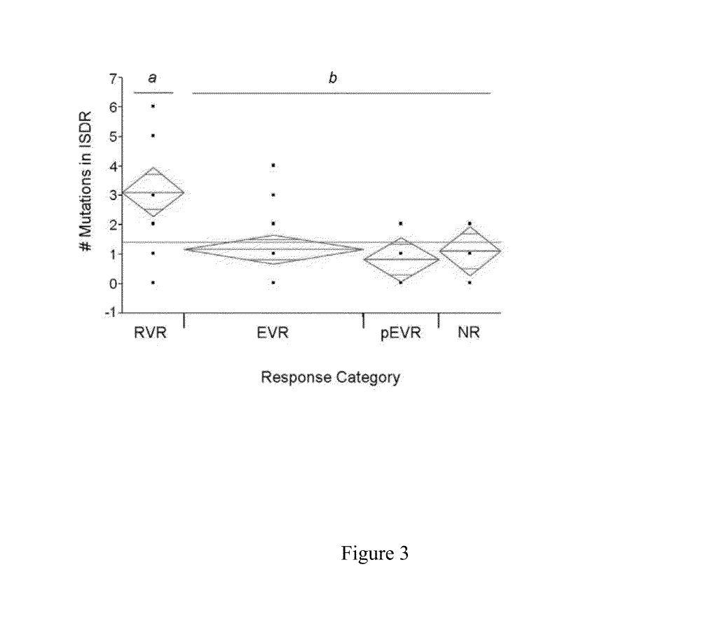

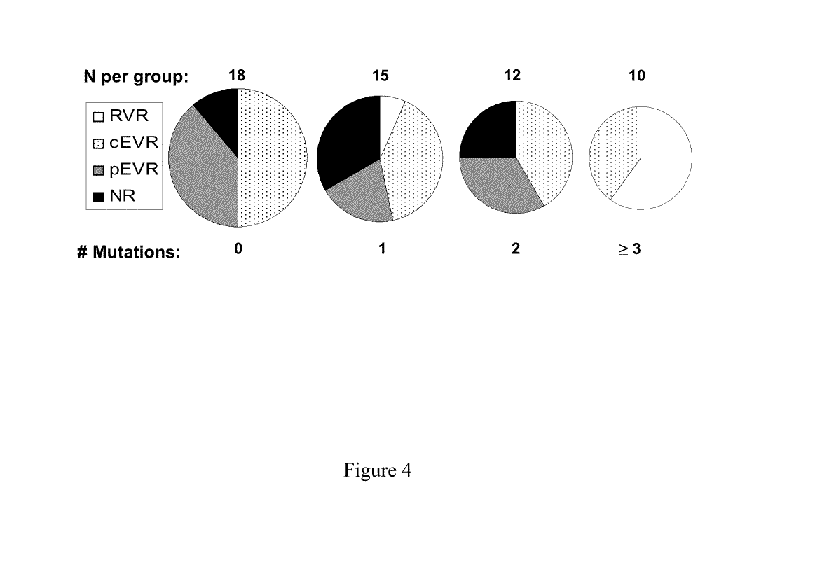

Within the ISDR, the RVR outcome group (median number of ISDR mutations per patient=3) had significantly more mutations than did the cEVR (median=1; Mann-Whitney U-test, p=0.0018), pEVR (median=0.5; Mann-Whitney U-test, p=0.0009), and NR (median=1; Mann-Whitney U-test, p=0.0031) groups. No significant differences were detected between any of the other outcome groups (FIG. 3). Only 1 patient with fewer than 3 mutations within the ISDR achieved an RVR, with all other RVR patients having at least 3 mutations within the ISDR (FIG. 4). All patients with 3 or more mutations in the ISDR (n=10) achieved either cEVR or RVR.

To identify other parameters which affect viral sensitivity to treatment with Peg-IFN and RBV, we developed a multivariate model utilizing patient demographic data (sex, race), initial viral load (range: 1.4×10⁵, 3.1×10⁷ IU/ml), and the amino acid composition of each residue in the NS5A protein. Additionally, the number of mutations within the ISDR was included as a predictor, given the univariate dependence of Peg-IFN and RBV sensitivity on this variable. The mixed multivariate ordinal logistic regression utilized forward and reverse selection, with significance level for entry set to 0.15 and the significance level threshold required for a variable to remain in the model set to 0.10. The results indicate that sex, and initial viral load do not affect PR sensitivity within our dataset. Interestingly, in addition to the number of mutations within the ISDR, changes within 2 of the 446 NS5A amino acid positions in NS5A were found to be correlated with IFN sensitivity: AA226 and AA311 (numbering based on HCV reference H77). In the case of AA226, methionine and glutamic acid were associated with Peg-IFN and RBV -sensitive phenotypes of HCV while alanine and leucine were associated with Peg-IFN and RBV -resistant phenotypes, with valine representing an intermediate phenotype. At position 311, glutamine, arginine, and alanine were associated with IFN-sensitivity whereas serine and proline were associated with Peg-IFN and RBV -resistance (Table 1).

TABLE 1

| | Non- | | | |
|---|---|---|---|---|
| Predictor | responsive | → | Responsive | p |
| AA 226 | A, L | V | M, E | <0.0001 |
| AA 311 | S, P | | Q, R, A | 0.0041 |
| ISDR | Few | | Many | 0.0002 |
| (# Mut.) | Mutations | | Mutations | |

Dependence of IFN sensitivity on the number of mutations within the ISDR and specific amino acid composition at 2 positions in NS5A allowed us to model patient responsiveness to Peg-IFN and RBV. When the model based on these three variables is applied to our dataset, the responses of 31 of 55 subjects (˜56%) are predicted accurately. Only 1 prediction was off by more than group, indicated by a non-responder (NR) predicted to be a cEVR.

In this study the investigators analyzed the full length NS5A protein from 55 genotype 1a American patients enrolled in the control arm of our PROVE1 (Phase 2) clinical trial, to determine regions that may confer a positive Peg-IFN and RBV response. Logistic regression was used to determine if sensitivity to Peg-IFN and RBV was a function of the number of variants within any region of the NS5A protein. Viral sensitivity to Peg-IFN and RBV was discovered to be a function of viral heterogeneity only within the ISDR (χ²=13.02, p=0.0003). Patients in the RVR outcome group had a significantly higher number of variants (median=3) in the ISDR when compared to the other treatment outcome groups (cEVR=1, pEVR=0.5 and NR=1). Our results contradict previous studies (Hofgartner 1997, Dal Pero 2007, and Murphy 2002) performed in genotype 1a patients, where investigators were unable to find a correlation between IFN sensitivity and the number of variants in the ISDR. Our results agree with Enomoto et al (1996) where subjects with high sequence variability in the ISDR were sensitive to therapy and patients whose sequence was identical to the consensus did not respond to therapy.

To identify other areas that may confer viral sensitivity to treatment with Peg-IFN and RBV, a multivariate model utilizing patient demographic data (sex, race), initial viral load and the amino acid composition of each residue in the NS5A protein as well as the number of variants in the ISDR were included as a predictor. Race, sex and initial viral load were included in the analysis due to their reported involvement in IFN response (Layden-Almer, Kemmer, Boulestin, Jessner, Nagaki, Dolin). These factors did not have an affect on IFN response due to a majority of the patients being of Caucasian descent and the baseline viral loads were within a 2 log range. With a larger and more diverse patient population these factors may have had a more pronounced affect on treatment response.

Results from the multivariate analysis identified not only the number of variants in the ISDR as conferring Peg-IFN and RBV sensitivity; it also identified two previously unreported residues AA226 and AA311. Patients with a methionine or a glutamic acid at residue 226 were associated with sensitivity to Peg-IFN and RBV, an alanine or leucine at this position resulted in a null response to Peg-IFN and RBV therapy. A glutamine, arginine or alanine at residue 311 was associated with Peg-IFN and RBV sensitivity whereas a serine or proline was associated with a Peg-IFN and RBV null response. Residue 226 was discovered to be within a highly conserved phosphorylation region downstream of the ISDR. This region contains the serine residues 224, 228 and 231 (aa 2197, 2201, 2204) which are needed for the hyperphosphorylation of NS5A (Tanji 1995). It is unclear what role NS5A hyperphosphorylation plays in the HCV life cycle, it has been suggested that HCV replication is regulated by the phosphorylation of NS5A (Koch 1999).

Another suggested function of NS5A, is modulation of host IFN stimulated antiviral responses, possibly mediated by NS5A interaction with. PKR (Gale 1997). The interaction between NS5A and PKR covers 66 residues in the center of NS5A (Koch 1999). Included in this interaction are two of the three serine residues needed for the hyperphosphorylation of NS5A, and flanking either side of this region are the novel residues 226 and 311. Whether phosphorylation of NS5A is needed in order to interact with PKR is unknown. It has been speculated that mutations outside the ISDR may influence cellular antiviral responses (Koch 1999). According to Sarasin-Filipowicz et al., patients who respond poorly to Peg-IFN and RBV therapy show a preactivation of their IFN system. This initial preactivation of the IFN system can be predictive of nonresponders thus making this patient population resistant to both endogenous IFN and IFN therapy (Sarasin-Filipowicz 2008).

Based on the analysis above, the investigators concluded that the virus most fit to withstand high basal IFN is the one with the following sequence signatures in NS5A: an alanine or leucine at residue 226, a serine or proline at residue 311 and <3 variants in the ISDR. Furthermore, it is concluded that patients with low basal IFN levels will respond well to Peg-IFN and RBV therapy because the virus was not under selective pressure within the host cell and when Peg-IFN and RBV therapy is introduced the virus is cleared. The sequence signatures for a patient with low basal IFN levels are: a methionine or glutamate at residue 226, glutamine, arginine or alanine at residue 311 and ≧3 variants in the ISDR. The investigators believe that by examining IFN levels prior to Peg-IFN and RBV therapy along with sequencing the NS5A region we would be able to predict the patient's response to therapy in order to determine the best course of treatment.

We claim:

1. A method for treating a patient infected with HCV-1a with interferon-based treatment comprising:
a) analyzing a partial or complete HCV NS5A gene of the patient; and
b) determining a criteria for predicting the likelihood of a positive response to the interferon-based treatment, wherein the criteria comprises one or more of the following elements:
i) the number of changes in the interferon sensitivity determining region (ISDR) of the patient's NS5A amino acid sequence when compared to a standard NS5A amino acid sequence; and
ii) the sequence of amino acid residue at position 226 of the patient's HCV NS5A amino acid sequence.

2. The method of claim 1 further including assigning weighting parameters for all the elements of the criteria under b) based on a sequence analysis of a population of HCV-1a infected patients and their respective response to the interferon-based treatment.

3. The method of claim 1, wherein the sequence of amino acid residue at position 226 of the patient's HCV NS5A amino acid sequence is A, L, V, E or M.

4. The method of claim 3, wherein the sequence of amino acid residue at position 226 of the patient's HCV NS5A amino acid sequence is M.

5. The method of claim 3, wherein the sequence of amino acid residue at position 226 of the patient's HCV NS5A amino acid sequence is E.

6. The method of claim 3, wherein the sequence of amino acid residue at position 226 of the patient's HCV NS5A amino acid sequence is A.

7. The method of claim 3, wherein the sequence of amino acid residue at position 226 of the patient's HCV NS5A amino acid sequence is L.

8. The method of claim 3, wherein the sequence of amino acid residue at position 226 of the patient's HCV NS5A amino acid sequence is V.

9. The method of any one of claims 1-8, wherein the criteria further includes an element of the sequence of amino acid residue at position 311 of the patient's HCV NS5A amino acid sequence, wherein the criteria comprises one or more of the three elements.

10. The method of claim 9 further including assigning weighting parameters to the element of the sequence of amino acid residue at position 311 of the patient's HCV NS5A amino acid sequence based on the sequence analysis of a population of HCV-1a infected patients and their response and their response to the interferon-based treatment.

11. The method of claim 9, wherein the sequence of amino acid residue at position 311 of the patient's HCV NS5A amino acid sequence is S, P, Q, R or A.

12. The method of claim 11, wherein the sequence of amino acid residue at position 311 of the patient's HCV NS5A amino acid sequence is S.

13. The method of claim 11, wherein the sequence of amino acid residue at position 311 of the patient's HCV NS5A amino acid sequence is P.

14. The method of claim 11, wherein the sequence of amino acid residue at position 311 of the patient's HCV NS5A amino acid sequence is Q.

15. The method of claim 11, wherein the sequence of amino acid residue at position 311 of the patient's HCV NS5A amino acid sequence is R.

16. The method of claim 11, wherein the sequence of amino acid residue at position 311 of the patient's HCV NS5A amino acid sequence is A.

17. The method of any one of claims 1-16 wherein the standard NS5A amino acid sequence is H77.

18. The method of claim 1 further including a step of analyzing a genetic polymorphism of the patient

19. The method of claim 18, wherein the genetic polymorphism of the patient is rs12979860.

20. The method of any one of claims 1-19, wherein the step of analyzing a partial or complete HCV NS5A gene of the patient includes a step of amplifying a portion of the partial or complete HCV NS5A gene using a polymerase chain reaction machine.

21. The method of any one of claims 1-19 further comprising a step of determining whether the patient responses positively to the interferon-based treatment.

22. The method of claim 20 further comprising a step of administering the patient the interferon-based treatment if the patient is determined to be responsive to the interferon-based treatment.

23. Use of interferon for the preparation of a medicament for the treatment of a patient infected with HCV-1a according a criteria for predicting the likelihood of a positive response to the interferon-based treatment, wherein the criteria comprises one or more of the following elements:,
a) the amino acid position 226 of the HCV NS5A amino acid sequence of the patient; and
b) the number of changes in the interferon sensitivity determining region in the NS5A amino acid sequence of the patient when compared to a standard NS5A amino acid sequence.

24. The use of claim 23, wherein the elements of the criteria are assigned weighting parameters based on a sequence analysis of a population of HCV-1a infected patients and their respective response to the interferon-based treatment.

25. The use of claim 23,wherein the amino acid position 226 of the NS5A amino acid sequence is A, L, V, M or E.

26. The use of claim 25, wherein the amino acid position 226 of the NS5A amino acid sequence is A.

27. The use of claim 25, wherein the amino acid position 226 of the NS5A amino acid sequence is L.

28. The use of claim 25, wherein the amino acid position 226 of the NS5A amino acid sequence is E.

29. The use of claim 25, wherein the amino acid position 226 of the NS5A amino acid sequence is M.

30. The use of claim 25, wherein the amino acid position 226 of the NS5A amino acid sequence is V.

31. The use of any one of claims 25-30, wherein the criteria further includes an element of the amino acid residue at position 311 of the NS5A amino acid sequence of the patient, wherein the criteria comprises one or more of the three elements.

32. The use of claim 31, wherein the amino acid residue at position 311 of the NS5A amino acid sequence of the patient is S, P, Q, R or A.

33. The use of claim 32, wherein in the NS5A amino acid sequence of the patient, the amino acid residue at position 311 is S.

34. The use of claim 32, wherein in the NS5A amino acid sequence of the patient, the amino acid residue at position 311 is P.

35. The use of claim 32, wherein in the NS5A amino acid sequence of the patient, the amino acid residue at position 311 is Q.

36. The use of claim 32, wherein in the NS5A amino acid sequence of the patient, the amino acid residue at position 311 is R.

37. The use of claim 32, wherein in the NS5A amino acid sequence of the patient, the amino acid residue at position 311 is A.

38. The use of any one of claims 24-37, wherein the medicament includes one or more anti-viral drugs.

39. The use of claim 38, wherein the one or more anti-viral drugs include ribavirin, a HCV protease inhibitor or a HCV polymerase inhibitor.

40. The use of claim 39, wherein the HCV protease inhibitor is BMS-790052, MK 7009, BI 201335, SCH900518, VX-985, SCHSO3034, VX-950, VX-500, R7227, ITMN-191, ACH-1095 or TMC435350.

41. The use of claim 39, wherein the HCV protease inhibitor is VX-950.

42. The use of claim 39, wherein the HCV protease inhibitor is SCHSO303.

43. The use of claim 39, wherein the HCV polymerase inhibitor is VCH-916, IDX-184, VX-222, filibuvir, ABT-033, ABT-072, GS190, ANA598, MK-3281, BMS-650032, or R7128.

44. The use of any one of claims 39-43, the medicament further includes a NS4A inhibitor, a NS4B inhibitor, Cyclophilin inhibitor and a combination thereof.

45. The use of any one of claims 38-44, the medicament further includes ACH-806, Clemizole, Delbio-025 or NIM811.

46. The use of any one of claims 23-45, wherein the standard NS5A amino acid sequence is H77.

47. The use of claim 23, wherein the criteria further includes a genetic polymorphism of the patient.
The method of claim 47, wherein the genetic polymorphism of the patient is rs12979860.

48. A method of prescribing a therapy regimen and/or duration for a patient infected with HCV-1a, comprising:
a) analyzing a partial or complete HCV NS5A gene of the patient; and
b) determining a criteria for predicting the likelihood of a positive response to the interferon treatment, wherein the criteria comprises one or more of the following elements:
i) the number of changes in the interferon sensitivity determining region of the patient's HCV NS5A amino acid sequence when compared to a standard NS5A amino acid sequence; and
ii) the sequence of amino acid residue at position 226 of the patient's HCV NS5A amino acid sequence; and
c) determining the therapy regimen and/or duration of the patient.

49. The method of claim 49 further including assigning weighting parameters for all the elements of the criteria under b) based on a sequence analysis of a population of HCV-1a infected patients and their respective response to the interferon-based treatment.

50. The method of claim 49 or 50, wherein the sequence of amino acid residue at position 226 of the patient's HCV NS5A amino acid sequence is A, L, V, E or M.

51. The method of claim 51, wherein the sequence of amino acid residue at position 226 of the patient's HCV NS5A amino acid sequence is A.

52. The method of claim 51, wherein the sequence of amino acid residue at position 226 of the patient's HCV NS5A amino acid sequence is L.

53. The method of claim 51, wherein the sequence of amino acid residue at position 226 of the patient's HCV NS5A amino acid sequence is M.

54. The method of claim 51, wherein the sequence of amino acid residue at position 226 of the patient's HCV NS5A amino acid sequence is E.

55. The method of claim 51, wherein the sequence of amino acid residue at position 226 of the patient's HCV NS5A amino acid sequence is V.

56. The method of any one of claims 49-56, wherein the criteria further includes an element of the sequence of amino acid residue at position 311 of the patient's HCV NS5A amino acid sequence, wherein the criteria comprises one or more of the three elements.

57. The method of any one of claims 48-56 further including assigning a weighting parameter to the element of the sequence of amino acid residue at position 311 of the patient's HCV NS5A amino acid sequence based on a sequence analysis of a population of HCV-1a infected patients and their respective response to the interferon-based treatment.

58. The method of claim 58, wherein the sequence of amino acid residue at position 311 of the patient's HCV NS5A amino acid sequence is S, P, Q, R or A

59. The method of claim 59, wherein the sequence of amino acid residue at position 311 of the patient's HCV NS5A amino acid sequence is S.

60. The method of claim 59, wherein the sequence of amino acid residue at position 311 of the patient's HCV NS5A amino acid sequence is P.

61. The method of claim 59, wherein the sequence of amino acid residue at position 311 of the patient's HCV NS5A amino acid sequence is Q.

62. The method of claim 59, wherein the sequence of amino acid residue at position 311 of the patient's HCV NS5A amino acid sequence is R.

63. The method of claim 59, wherein the sequence of amino acid residue at position 311 of the patient's HCV NS5A amino acid sequence is A.

64. The method of any one of claims 49-64, wherein the standard NS5A amino acid sequence is H77.

65. The method of any one of claims 49-65, wherein determining a regimen and/or duration of the patient's therapy comprises administering the patient a HCV-protease inhibitor, a second STAT-C, interferon, ribavirin or a combination thereof.

66. The method of claim 66, wherein administering the patient comprising administering the patient for a 12-week, 36-week or 48-week duration

67. The method of claim 66, wherein administering the patient comprising administering the patient a 12-week duration.

68. The method of claim 66, wherein administering the patient comprising administering the patient a 36-week duration.

69. The method of claim 66, wherein administering the patient comprising administering the patient for a 48-week duration.

70. The method of any one of claims 66-70, wherein the HCV protease inhibitor is SCHSO3034, VX-950, VX-500, R7227, ITMN-191, ACH-1095 or TMC435350.

71. The method of any one of claims 71, wherein the HCV protease inhibitor is SCHSO3034.

72. The method of any one of claims 71, wherein the HCV protease inhibitor is VX-950.

73. The method of claim 66, wherein the second STAT-C is a HCV polymerase inhibitor, a NS4A inhibitor, a NS4B inhibitor or Cyclophilin inhibitor.

74. The method of claim 74, wherein the second STAT-C is VCH-916, IDX-184, VX-222, filibuvir, ABT-033, ABT-072, GS190, ANA598, MK-3281, BMS-650032, ACH-806, Clemizole, Delbio-025, NIM811 or R7128.

75. The method of claim 49 further including a step of analyzing a genetic polymorphism of the patient.

76. The method of claim 76, wherein the genetic polymorphism of the patient is rs12979860.

77. The method of any one of claims 49-77, wherein the step of analyzing a partial or complete HCV NS5A gene of the patient includes a step of amplifying a portion of the partial or complete HCV NS5A gene using a polymerase chain reaction machine.

78. The method of any one of claims 49-78 further comprising a step of administering the patient the interferon-based treatment.


SEQUENCE LISTING (13 sequences)

SEQ ID NO: 1 - DNA, 601 bp, Homo sapiens
caggactgcggggacgagagggcgttagagcgggccgcgcccgggccatgcctctcccgcccactcccgggcctcaccgatggccgcggaggatccctcctggggcggaaggagcagttgcgctgcccccagctcagcgcctcttcctcctgcgggacaagcggcgcttatcgcatacggctaggccccctcgccagggcccctaacctctgcacagtctgggattcctggacgtggatgggtactggcagcgcacggtcgtgcctgtcgtgtactgaaccagggagctccccgaaggcgygaaccagggttgaattgcactccgcgctcccccagcaaagcccctcgccccgacctggagccgagtcctcccggcagggctcccttctgtgattgaccctgagcctgcgttcgcgctgacgacggggactgcgggggtctcgtggtgggaattgtgggcgctgacataggagaggcgcctgctgggcgctaggacgcaggaccccttgggacaggaacgggtgtatgggaacccggtggggccagggtcccagggggcacaggggctgggcggtgacttacgtagcggtccctcagcgcc

SEQ ID NO: 2 - DNA, 1338 bp, Hepatitis C virus
tccggttcctggctaagggacatctgggactggatatgcgaggtgctgagcgactttaagacctggctgaaagccaagctcatgccacaactgcctgggattccctttgtgtcctgccagcgcgggtataggggggtctggcgaggagacggcattatgcacactcgctgccactgtggagctgagatcactggacatgtcaaaaacgggacgatgaggatcgtcggtcctaggacctgcaggaacatgtggagtgggacgttccccattaacgcctacaccacgggcccctgtactccccttcctgcgccgaactataagttcgcgctgtggagggtgtctgcagaggaatacgtggagataaggcgggtgggggacttccactacgtatcgggtatgactactgacaatcttaaatgcccgtgccagatcccatcgcccgaatttttcacagaattggacggggtgcgcctacacaggtttgcgcccccttgcaagcccttgctgcgggaggaggtatcattcagagtaggactccacgagtacccggtggggtcgcaattaccttgcgagcccgaaccggacgtagccgtgttgacgtccatgctcactgatccctcccatataacagcagaggcggccgggagaaggttggcgagagggtcacccccttctatggccagctcctcggctagccagctgtccgctccatctctcaaggcaacttgcaccgccaaccatgactcccctgacgccgagctcatagaggctaacctcctgtggaggcaggagatgggcggcaacatcaccagggttgagtcagagaacaaagtggtgattctggactccttcgatccgcttgtggcagaggaggatgagcgggaggtctccgtacctgcagaaattctgcggaagtctcggagattcgcccgggccctgcccgtctgggcgcggccggactacaaccccccgctagtagagacgtggaaaaagcctgactacgaaccacctgtggtccatggctgcccgctaccacctccacggtcccctcctgtgcctccgcctcggaaaaagcgtacggtggtcctcaccgaatcaaccctatctactgccttggccgagcttgccaccaaaagttttggcagctcctcaacttccggcattacgggcgacaatacgacaacatcctctgagcccgccccttctggctgcccccccgactccgacgttgagtcctattcttccatgccccccctggagggggagcctggggatccggatctcagcgacgggtcatggtcgacggtcagtagtggggccgacacggaagatgtcgtg

SEQ ID NO: 3 - protein, 446 aa, Hepatitis C virus
SGSWLRDIWDWICEVLSDFKTWLKAKLMPQLPGIPFVSCQRGYRGVWRGDGIMHTRCHCGAEITGHVKNGTMRIVGPRTCRNMWSGTFPINAYTTGPCTPLPAPNYKFALWRVSAEEYVEIRRVGDFHYVSGMTTDNLKCPCQIPSPEFFTELDGVRLHRFAPPCKPLLREEVSFRVGLHEYPVGSQLPCEPEPDVAVLTSMLTDPSHITAEAAGRRLARGSPPSMASSSASQLSAPSLKATCTANHDSPDAELIEANLLWRQEMGGNITRVESENKVVILDSFDPLVAEEDEREVSVPAEILRKSRRFARALPVWARPDYNPPLVETWKKPDYEPPVVHGCPLPPPRSPPVPPPRKKRTVVLTESTLSTALAELATKSFGSSSTSGITGDNTTTSSEPAPSGCPPDSDVESYSSMPPLEGEPGDPDLSDGSWSTVSSGADTEDVV

SEQ ID NO: 4 - DNA, 1338 bp, Hepatitis C virus
tccggttcttggctaagggacatctgggactggatatgcgaggtgctgagcgattttaagacctggcttaaggccaagctcacgccacaactgcctgggattccctttgtgtcctgccaacgcgggtataggggggtctggcgcggagacggcattatgcacactcgttgccgctgcggagctgacatcactggacatgtcaagaacgggacgatgaggatcgttggtcctaggacctgcaggaacatgtggagtggaaccttccccatcaacgcctacaccacgggcccctgtacccccctccctgcgccgaactatacgttcgcgctktggagggtggctgcggaggaatacgtggaaataaggcgggtgggggacttccactacgtgacgggcatgaccactgacaatctcaagtgcccatgccaggtcccatcgcccgaatttttcacagaactggacggggtgcgcctgcacaggtatgcgcccccttgcaagcctttgctacgggatgaggtgtcgttcaaagtaggactacacgattacccggtggggtcgcaattaccttgcgaacctgaactggacgtggccgtggtgacgtccatgctcactgacccctcccatataacggcagaggcggctaggaggaggctggcaaggggatcccccccttctgaggccagctcctcggccagccagctgtccgctccatctctcaaggcaacctgtccaaccaaccacgactcccctgacgccgagctcgtagaggctgacctcctgtggtggcgggagatgggcggcaacatcaccagggttgagtcagagaacaaagtggtgattctggactcttttgatccgcttgtggcggaggagaatgaacgggaggtctccgtgcccgcggagatcctgcggaagtctcggagattcgccccggccctgcccatttgggcacggccggactacaaccccccgttgctggagacgtggaaaaagccggactacgaaccacctgtggtccacggctgcccgcttccacctccacagtctcctcctgtgcctccgccccggaaaaagcggacggtggtcctcaccgaatcaacggtatctactgccttggccgagcttgccactaagacctttggcagctcctcaactcccggcattatgggtggcgatacatcgacgccctctgagctcgccccccctgcctgccctccagactccgacgccgagtcctgttcctccatgccccccctagagggggagcctggggatccggacctcagcgacgggtcatggtcgacgattagcggtggggctgatacggaggatgtcgtg

SEQ ID NO: 5 - DNA, 1338 bp, Hepatitis C virus
tccggttcctggctgagggacatctgggactggrtatgcgaggtgctgagcgacttcaagacctggctraragccaagctcatgccacaactgcctgggattccccttgtgtcctgtcagcgcgggtatargggggtctggcgaggggatggcatcatgcacactcgctgcaactgtggggctgagatyactggacatgtcaaaaacgggacgatgaggatcgtcggtcctaggacctgcaggaacatgtggagcgggaccttccccattaatgcctacaccacaggcccctgtgtccccctccccgcgccgaactacacgtttgcgctgtggagggtgtccgcagaggaatacgtggagataaggcgggtgggagacttccactatgtgacgggtatgactactgacaaccttaaatgcccgtgccaggtcccgtcgcccgaatttttcacagaattggacggggtgcgcctacataggtttgcgcccccctgcaagcccttgctgcgggaggawgtatcattyagagtaggactccacgaatacccggtggggtcgcagttgccctgcgagcccgaaccggatgtggccgtgttgacgtccatgctcactgatccctcccatataacagcagaggcggccgggagaaggttggcgaggggatcacccccttctgtggccagctcctcggctagccagctatctgctccatccmtcaaggcaacttgcaccgtcaaccatgactcccctgacgccgamctcatagaagctaacctcctatggaggcaggagatgggcggcaacatcaccagggtcgagtcagagaacaaagtggtggttctggactccttcgatccgctcgtggcggaggaggacgagcgggagatctccgtacccgcagagatcctgcggaagtctcggaggttcrcccaggccytgcctatttgggcgcggccggactataaccccccgctattggagacgtggaaaaagcctgactacgaaccacctgtggtccatggctgtccgcttccacctccacagtcccctcctgtgcctccgccccggaagaagcggacggtggtcctcaccgaatcaacyctatctactgccttggccgagcttgccaccaagagctttggyagcccctcaacttccggtgttacgggtgacgatacgacaacatcttctgagcccgcctcttctggccgcccccccgactccgacactgagtcctattcttccatgccccccctggagggggagccsggggatccggatctcagcgacgggtcatggtcgacggtcagtagtgaggccggtacggaggatgtcgtg

SEQ ID NO: 6 - DNA, 1338 bp, Hepatitis C virus
tccgattcctggctaagggacatctgggactggatatgcgaagtgctgagcgactttaagacctggctaaaagccaagctcatgccacaactgcctgggattccctttgtgtcctgccagcgcgggtataggggggtctggcgaggggatggcatyatgcacactcgctgccactgtggagctgagatcaccggacatgtcaaaaacgggacgatgaggatcgtcggcccgaggacctgcaggaacatgtggagtggaaccttccccattaacgcctacacaacgggtccctgtacccccctccccgcgccaaactacaagttcgcgctgtggagggtgtctgcagaggartacgtggagataaggcaagtgggggatttccactacgtgacgggtatgactactgacaaccttaaatgcccgtgccaggtcccatcgcccgaatttttcacagaattggacggggtgcgcctacataggtttgcgcccccctgcaagcccttgctgcgggatgaggtgtcattcagagtaggactccacgcgtacccggtggggtcgcaattaccttgcgaacccgaaccggacgtggccgtgttgacgtccatgctcactgatccttcccatataacggcagaggcggccgggagaaggttggcgaggggatcacccccttccatggccagctcctcggctagccagctgtccgctccatctctcaaggcaacttgcaccgccaaccatgactcccctgatgccgagctcttagaggctaacctcctatggaggcaagagatgaacggcaacatcaccagggttgagtcagagaacaaagtggtgattctggactccttcgaaccgctcgtggcggaggaggatgagcgggagatctccgtacccgcggaattgctgcggaagtctcggagattcaccccggccctgcccgtttgggcgcggccggactataaccccccgctagtggaggtgtggaaaaagcccgactacgagccacctgtggtccatggctgcccgcttccacctccacggtcccctcctgtgcctccgcctcggaagaagcggacggtggtcctcaccgaatcaaccgtatctactgccttggccgagcttgccaccaagagttttggcagctcctcaacttccggcatcacgggcagcaacatgacagagtcctctgagcccgcccctcctggctgccccccggactccgacgctgagtcatattcttccatgccccccctggagggggagcctggggatccggatctcagcgacgggtcgtggtcgacggtcagtagtgggtccggcacggaggatgtcgtg

SEQ ID NO: 7 - DNA, 1338 bp, Hepatitis C virus
accggttcctggctaagagacatctgggattggatatgcgaggtgctgagcgactttaagacctggctgaaagccaagctcatgccacaactgcctgggatcccctttgtgtcctgccarcgcgggtataagggggtctggcggggggacggcgtcatgcacactcgctgccactgtggagctgagatcactggacatgtcaagaacgggacgatgaggatcgtcggtcctaagacctgcaggaacatgtggagtgggaccttccccattaacgcctacacaacgggcccctgtacycccctccccgcgccgaactacacgttcgcgctgtggagggtgtctgcagaggagtacgtggagataaggcgggtgggggacttccactacgtgacgggcatgactactgacgatcttaaatgcccgtgccaggtcccatctcccgaatttttcacagaattggacggggtgcgcctacataggtttgcgcccccctgcaagcccttgctgcgggatgaggtatcattcagagtaggactccacgcgtacccggtggggtcgcaattaccttgcgagccygaaccggatgtggccgtgttgacgtccatgctcactgacccctcccatataacagcagaggcggccgggagaaggttggcgaggggatcacccccttctgtggccagctcctcggctagccagctgtctgctccatctctcaaggcaacttgcaccaccaaccatgactcccctgatgccgagctcatagaggctaacctcctatggaggcaagagatgggcggcaacatcaccagggttgagtcagagaacaaagtggtgattctggactccttcgatccgcttgtggcggaggaggatgagcgggagatctccgtacccgcrgaaatcctgcggaagtctcggagattcgccccagccctgcccrtttgggcgcggccggactayaaccccccgctaktggagacgtggaaaaagcctgactacgagccacctgtggtccatggctgcccgcttccacctccacagtcccctcctgtgcctccgcctcggaagaagcgtacggyggtcctcaccgaatcaaccgtatcctctgccttggccgagcttgccaccaaaagctttggcagctcctcaacttccggaattacgggcgacaacacgacaacatcctctgagcccgccccttctggctgcctccctgactccgacgctgagtcctattcttccatgccccccctggagggggagcccggggatccggatctcagcgacgggtcgtggtcgacggtcagtagtgaggccggcacagaggatgtcgtg

SEQ ID NO: 8 - DNA, 1338 bp, Hepatitis C virus
tccggctcctggttaagggacatctgggactgggtatgcgaggtgctgagcgaytttaagacctggctgaaggccaagctcgtgccacacctgcctgggattccctttgtatcctgccaacgcgggtataggggggtctggcgaggggatggcatcatgcacactcgctgccactgcggagctgagatcgctggacatgtcaaaaacgggacgatgaggatcgtcggtcctaagacctgcaggaacayrtggagtgggaccttccccatcaacgcctacaccacgggcccctgyacccccctccctgcgccgaactatacgttcgcgctrtggagggtgtctgcrgaggaatacgtggaaataaggcaggtgggggacttccactacgtgacgggcgtgactactgacaatcttaratgcccatgccaggtcccatcacccgaatttttcacagaactggacggggtgcgcctrcataggtttgcgcccccttgcaarcctctgctacgggatgaggtgtcgttcagagtaggactacaygattacccggtggggtcgcaaytaccttgcgagccygaaccggacgtggccgtgttgacgtccatgctcactgacccttcccatataacagcagaggcggctgggaggaggttggcaaggggatcgcccccgtctttggccagytcctcggccagccagctgtccgctccatctctcaaggcaacttgcaccaccaaccatgactcycctgacgccgagctcattgaggctaatctcctgtggaggcaggagatgggtggtaacatcaccagggttgagtcggagaacaaagtggtggttctggactccttcgatccgcttgtggcrgaggaggatgaacgggaggtttccgtgcccgcrgaaatcctgcggaagactcggaaattcaccmcggccctgcccgtttgggcacggccggactacaaccccccgttrctggagacgtggaaaaarccggactacgaaccacctgtggtccayggctgcccacttccacctccacrgtcccctcctgtgccgccgccccggaagaagcggacggtggtcctctcwgaatcaaccgtgtccaccgcyttggccgagcttgccaccaagagctttggcagcccctcaacttccggtgtcacgggcgacratacaacgacgtcctctgagcccgccccctctgtctgycctccagactccgaygctgagtcctattcttccatgccccccctggagggggagcctggggatccggayctcagygacgggtcatggtcgacggttagtagtgaggctggcacggaggatgtygtg

SEQ ID NO: 9 - protein, 446 aa, Hepatitis C virus
SGSWLRDIWDWICEVLSDFKTWLKAKLTPQLPGIPFVSCQRGYRGVWRGDGIMHTRCRCGADITGHVKNGTMRIVGPRTCRNMWSGTFPINAYTTGPCTPLPAPNYTFALWRVAAEEYVEIRRVGDFHYVTGMTTDNLKCPCQVPSPEFFTELDGVRLHRYAPPCKPLLRDEVSFKVGLHDYPVGSQLPCEPELDVAVVTSMLTDPSHITAEAARRRLARGSPPSEASSSASQLSAPSLKATCPTNHDSPDAELVEADLLWWREMGGNITRVESENKVVILDSFDPLVAEENEREVSVPAEILRKSRRFAPALPIWARPDYNPPLLETWKKPDYEPPVVHGCPLPPPQSPPVPPPRKKRTVVLTESTVSTALAELATKTFGSSSTPGIMGGDTSTPSELAPPACPPDSDAESCSSMPPLEGEPGDPDLSDGSWSTISGGADTEDVV

SEQ ID NO: 10 - protein, 446 aa, Hepatitis C virus
  MOD_RES (12)..(12): Any amino acid
SGSWLRDIWDWXCEVLSDFKTWLXAKLMPQLPGIPLVSCQRGYXGVWRGDGIMHTRCNCGAEITGHVKNGTMRIVGPRTCRNMWSGTFPINAYTTGPCVPLPAPNYTFALWRVSAEEYVEIRRVGDFHYVTGMTTDNLKCPCQVPSPEFFTELDGVRLHRFAPPCKPLLREXVSFRVGLHEYPVGSQLPCEPEPDVAVLTSMLTDPSHITAEAAGRRLARGSPPSVASSSASQLSAPSXKATCTVNHDSPDAXLIEANLLWRQEMGGNITRVESENKVVVLDSFDPLVAEEDEREISVPAEILRKSRRFXQALPIWARPDYNPPLLETWKKPDYEPPVVHGCPLPPPQSPPVPPPRKKRTVVLTESTLSTALAELATKSFGSPSTSGVTGDDTTTSSEPASSGRPPDSDTESYSSMPPLEGEPGDPDLSDGSWSTVSSEAGTEDVV

SEQ ID NO: 11 - protein, 446 aa, Hepatitis C virus
SDSWLRDIWDWICEVLSDFKTWLKAKLMPQLPGIPFVSCQRGYRGVWRGDGIMHTRCHCGAEITGHVKNGTMRIVGPRTCRNMWSGTFPINAYTTGPCTPLPAPNYKFALWRVSAEEYVEIRQVGDFHYVTGMTTDNLKCPCQVPSPEFFTELDGVRLHRFAPPCKPLLRDEVSFRVGLHAYPVGSQLPCEPEPDVAVLTSMLTDPSHITAEAAGRRLARGSPPSMASSSASQLSAPSLKATCTANHDSPDAELLEANLLWRQEMNGNITRVESENKVVILDSFEPLVAEEDEREISVPAELLRKSRRFTPALPVWARPDYNPPLVEVWKKPDYEPPVVHGCPLPPPRSPPVPPPRKKRTVVLTESTVSTALAELATKSFGSSSTSGITGSNMTESSEPAPPGCPPDSDAESYSSMPPLEGEPGDPDLSDGSWSTVSSGSGTEDVV

SEQ ID NO: 12 - protein, 446 aa, Hepatitis C virus
  MOD_RES (315)..(315): Any amino acid
TGSWLRDIWDWICEVLSDFKTWLKAKLMPQLPGIPFVSCQRGYKGVWRGDGVMHTRCHCGAEITGHVKNGTMRIVGPKTCRNMWSGTFPINAYTTGPCTPLPAPNYTFALWRVSAEEYVEIRRVGDFHYVTGMTTDDLKCPCQVPSPEFFTELDGVRLHRFAPPCKPLLRDEVSFRVGLHAYPVGSQLPCEPEPDVAVLTSMLTDPSHITAEAAGRRLARGSPPSVASSSASQLSAPSLKATCTTNHDSPDAELIEANLLWRQEMGGNITRVESENKVVILDSFDPLVAEEDEREISVPAEILRKSRRFAPALPXWARPDYNPPLXETWKKPDYEPPVVHGCPLPPPQSPPVPPPRKKRTXVLTESTVSSALAELATKSFGSSSTSGITGDNTTTSSEPAPSGCLPDSDAESYSSMPPLEGEPGDPDLSDGSWSTVSSEAGTEDVV

SEQ ID NO: 13 - protein, 446 aa, Hepatitis C virus
  MOD_RES (83)..(83): Any amino acid
SGSWLRDIWDWVCEVLSDFKTWLKAKLVPHLPGIPFVSCQRGYRGVWRGDGIMHTRCHCGAEIAGHVKNGTMRIVGPKTCRNXWSGTFPINAYTTGPCTPLPAPNYTFALWRVSAEEYVEIRQVGDFHYVTGVTTDNLXCPCQVPSPEFFTELDGVRLHRFAPPCKPLLRDEVSFRVGLHDYPVGSQLPCEPEPDVAVLTSMLTDPSHITAEAAGRRLARGSPPSLASSSASQLSAPSLKATCTTNHDSPDAELIEANLLWRQEMGGNITRVESENKVVVLDSFDPLVAEEDEREVSVPAEILRKTRKFTXALPVWARPDYNPPLLETWKKPDYEPPVVHGCPLPPPXSPPVPPPRKKRTVVLSESTVSTALAELATKSFGSPSTSGVTGDXTTTSSEPAPSVCPPDSDAESYSSMPPLEGEPGDPDLSDGSWSTVSSEAGTEDVV